(19) United States
(12) Patent Application Publication
(10) Pub. No.: US 2025/0090562 A1
(43) Pub. Date: Mar. 20, 2025

(54) METHODS, COMPOSITIONS AND USES RELATED TO THE TREATMENT AND PREVENTION OF LEIGH’S SYNDROME

(71) Applicant: Stealth BioTherapeutics Inc., Needham, MA (US)

(72) Inventors: David Brown, Needham, MA (US); Hatim Zariwala, Needham, MA (US); Laura Kropp, Needham, MA (US); Alyssa Handler, Needham, MA (US)

(73) Assignee: Stealth BioTherapeutics Inc., Needham, MA (US)

(21) Appl. No.: 18/963,207

(22) Filed: Nov. 27, 2024

Related U.S. Application Data
(63) Continuation of application No. PCT/US2024/038657, filed on Jul. 19, 2024.
(60) Provisional application No. 63/528,186, filed on Jul. 21, 2023.
(60) Provisional application No. 63/606,826, filed on Dec. 6, 2023.

(51) Int. Cl. A61K 31/695; A61K 31/122

(52) U.S. Cl. CPC A61K 31/695; A61K 31/122

(57) ABSTRACT

The present disclosure provides methods for treating, preventing, ameliorates, inhibits, or delaying the onset of Leigh syndrome as well as methods for ameliorating, inhibiting, ameliorates, inhibits, or delaying the onset of its associated signs and/or symptoms in a subject in need thereof comprising administering to the subject various compounds, mixtures of compounds or compositions, formulations or medicaments derived therefrom. The present disclosure further provides compositions, formulations or medicaments and related uses for the treatment, prevention ameliorating, inhibiting, or delaying the onset of Leigh syndrome in a subject and addressing its associated signs and/or symptoms.

### CROSS-REFERENCE TO RELATED APPLICATIONS

This application claims the benefit of and priority to U.S. Provisional Patent Application No. 63/528,186, filed Jul. 21, 2023, and U.S. Provisional Patent Application No. 63/606,826, filed Dec. 6, 2023, the entire contents of each of which are incorporated herein by reference for any and all purposes.

### TECHNICAL FIELD

The present application relates generally to methods, compositions/formulations/medicaments and related uses for treating, preventing, ameliorating, inhibiting, or delaying the onset of Leigh syndrome, in a subject in need thereof and/or its associated signs and/or symptoms in said subject. The methods, compositions/formulations/medicaments and related uses all relate to the administration of one or more of four compounds specifically identified herein.

### INTRODUCTION

The following description is provided to assist the understanding of the reader. None of the information provided or references cited herein is admitted as being prior art to the compounds, compositions, products and/or methods disclosed herein.

Leigh syndrome (also known as Leigh disease) is a rare neurodegenerative disease associated with mitochondrial dysfunction that generally begins in infants between the ages of three months and two years. In rare cases, the onset of the disease or its signs and symptoms is delayed into the teen years or even into adulthood. For children with Leigh syndrome, the disease is usually rapidly progressive. Children with early-onset Leigh syndrome often pass away by age three and 90% of deaths often occur by age 6. Children with Leigh syndrome often die of respiratory failure. People with adult-onset Leigh syndrome can live past their fifties.

Early signs and symptoms of Leigh syndrome include loss of appetite, poor sucking ability, vomiting, irritability, continuous crying, and seizures. The disease then usually progresses to generalized weakness, lack of muscle tone (including loss of head control and motor skills), and episodes of lactic acidosis, which can lead to impairment of respiratory and kidney function. Clinical symptoms include neurodevelopmental deterioration, which is often accompanied by brainstem dysfunction including abnormalities in tone, power, reflexes, ataxia, dysphagia, and seizures. Other signs or symptoms of the disease include difficulty swallowing, difficulty breathing, difficulty eating, hypotonia, developmental delays, ataxia, dysarthria, dystonia, and paralysis. While the clinical presentations might differ between individuals, Leigh syndrome symptoms are significantly impacted by abnormalities in the brainstem, cerebellum, basal ganglia, oculomotor and cranial nerves. Because Leigh syndrome involves mitochondrial dysfunction, and because the heart is mitochondria rich, cardiomyopathies (e.g., hypertrophic cardiomyopathy and dilated cardiomyopathy) are often also seen in patients with Leigh syndrome. The patient may also experience vision impairments (e.g., color blindness or vision loss).

Vatiquinone (also known as EPI743) has been tested in the clinic as a possible treatment for Leigh syndrome but recent results appear to be disappointing. Vatiquinone is believed to target ferroptosis in treated subjects (See: Kahn-Kirby et al, Targeting ferroptosis: A novel therapeutic strategy for treatment of mitochondrial disease-related epilepsy). Treatments generally are directed to treating signs and symptoms of the disease. Better treatments are needed to address this devastating rare disease.

### SUMMARY

In one aspect, the present technology provides a method for treating, preventing, ameliorating, inhibiting, or delaying the onset of Leigh syndrome, or its associated signs and/or symptoms, in a subject in need thereof, comprising administering to said subject a therapeutically effective amount of Compound 1, Compound 2, Compound 3, Compound 4, or a mixture of any two or more of Compounds 1 to 4, or pharmaceutically acceptable salts, stereoisomers, mixtures of stereoisomers, tautomers, hydrates, and/or solvates thereof, wherein Compounds 1 to 4 have the following structures:

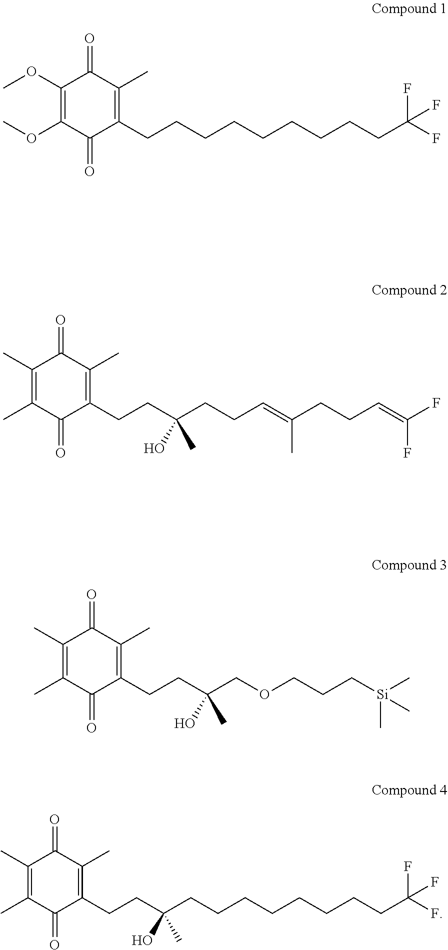

In some embodiments, the signs or symptoms of Leigh syndrome include difficulty swallowing, difficulty breathing, difficulty eating, hypotonia, lactic acidosis, developmental delays, cardiomyopathy, seizures, ataxia, dysarthria, dystonia, paralysis, impairment of respiratory and kidney function, color blindness and/or vision loss.

In some embodiments, the subject has been diagnosed with Leigh syndrome. In some embodiments, the subject is human.

In some embodiments, the Compound(s) is/are administered daily for 6 weeks or more.

In some embodiments, the Compound(s) is/are administered orally, topically, intranasally, systemically, intravenously, subcutaneously, intraperitoneally, intradermally, intraocularly, ophthalmically, intrathecally, intracerebroventricularly, iontophoretically, transmucosally, intravitreally, or intramuscularly.

In some embodiments, administration of the Compound(s), to the subject treats, prevents, ameliorates, inhibits, or delays the onset of a cardiomyopathy in the subject.

In some embodiments, the cardiomyopathy is hypertrophic cardiomyopathy, left ventricular hypertrophy, dilated cardiomyopathy, pericardial effusions and/or an arrhythmia/conduction abnormality. In some embodiments, the cardiomyopathy is hypertrophic cardiomyopathy.

In some embodiments, administration of the Compound(s) to the subject lowers lactic acid levels in the subject's blood, urine, or cerebrospinal fluid (CSF).

In some embodiments, administration of the Compound(s), to the subject, treats, prevents, ameliorates, inhibits, or delays the onset of multi-organ and/or central nervous system (CNS) damage, including lesions in the brainstem, basal ganglia, and spinal cord of the subject.

In some embodiments, administration of the Compound(s) to the subject treats, prevents, ameliorates, inhibits, or delays the onset of color blindness and/or vision loss of the subject.

In some embodiments, administration of the Compound(s), to the subject, increases the lifespan of the subject.

In some embodiments, administration of the Compound(s) to the subject, treats, prevents, ameliorates, inhibits, or delays the onset of seizures, ataxia, dysarthria, dystonia and/or paralysis of the subject.

In one aspect, the present technology provides a composition, medicament or formulation comprising any one of Compounds 1 to 4, or a mixture of any two or more of Compounds 1 to 4, or pharmaceutically acceptable salts, stereoisomers, mixtures of stereoisomers, tautomers, hydrates, and/or solvates thereof for use in treating, preventing, ameliorating, inhibiting, or delaying the onset of Leigh syndrome, or its associated signs and/or symptoms in a subject in need thereof, wherein Compounds 1 to 4 have the following structures:

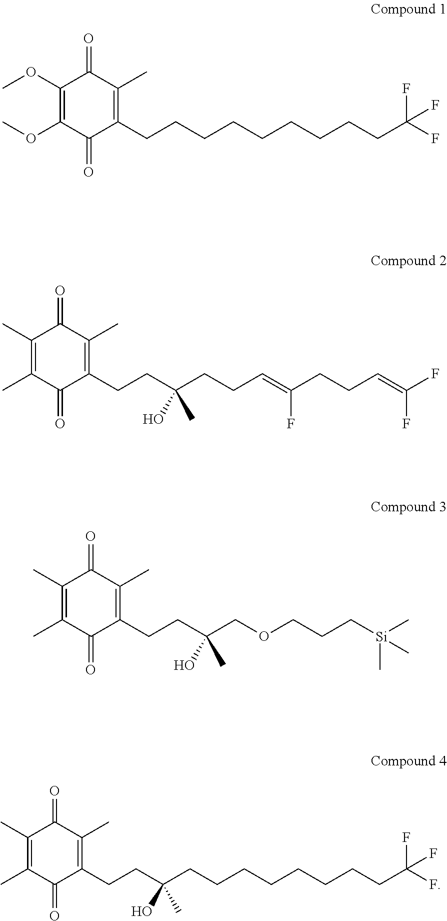

In some embodiments, the signs or symptoms of Leigh syndrome include difficulty swallowing, difficulty breathing, difficulty eating, hypotonia, lactic acidosis, developmental delays, cardiomyopathy, seizures, ataxia, dysarthria, dystonia, paralysis, impairment of respiratory and kidney function, color blindness and vision loss.

In some embodiments, the subject has been diagnosed with Leigh syndrome. In some embodiments, the subject is human.

In some embodiments, the composition, medicament or formulation is administered daily for 6 weeks or more.

In some embodiments, the composition, medicament or formulation is administered orally, topically, intranasally, systemically, intravenously, subcutaneously, intraperitoneally, intradermally, intraocularly, ophthalmically, intrathecally, intracerebroventricularly, iontophoretically, transmucosally, intravitreally, or intramuscularly.

In some embodiments, administration of the composition, medicament, or formulation, to the subject treats, prevents, ameliorates, inhibits, or delays the onset of a cardiomyopathy in the subject.

In some embodiments, the cardiomyopathy is hypertrophic cardiomyopathy, left ventricular hypertrophy, dilated cardiomyopathy, pericardial effusions and/or an arrythmia/conduction abnormality. In some embodiments, the cardiomyopathy is hypertrophic cardiomyopathy.

In some embodiments, administration of the composition, medicament, or formulation to the subject lowers lactic acid levels in the subject's blood, urine, or cerebrospinal fluid (CSF).

In some embodiments, administration of the composition, medicament, or formulation, to the subject treats, prevents, ameliorates, inhibits, or delays the onset of multi-organ and/or central nervous system (CNS) damage, including lesions in the brainstem, basal ganglia, and spinal cord of the subject.

In some embodiments, administration of the composition, medicament, or formulation, to the subject treats, prevents, ameliorates, inhibits, or delays the onset of color blindness and/or vision loss of the subject.

In some embodiments, administration of the composition, medicament, or formulation, to the subject, increases the lifespan of the subject.

In some embodiments, administration of the composition, medicament, or formulation, to the subject treats, prevents, ameliorates, inhibits, or delays the onset of seizures, ataxia, dysarthria, dystonia and/or paralysis of the subject.

In one aspect, the present technology provides a use of Compounds 1 to 4, or a mixture of any two or more of Compounds 1 to 4, or pharmaceutically acceptable salts, stereoisomers, mixtures of stereoisomers, tautomers, hydrates, and/or solvates thereof in the preparation of a composition, medicament or formulation comprising any one of Compounds 1 to 4, or a mixture of any two or more of Compounds 1 to 4, or pharmaceutically acceptable salts, stereoisomers, mixtures of stereoisomers, tautomers, hydrates, and/or solvates thereof suitable for treating, preventing, ameliorating, inhibiting, or delaying the onset of Leigh syndrome, or the signs or symptoms of Leigh syndrome, in a subject in need thereof, wherein Compounds 1 to 4 have the following structures:

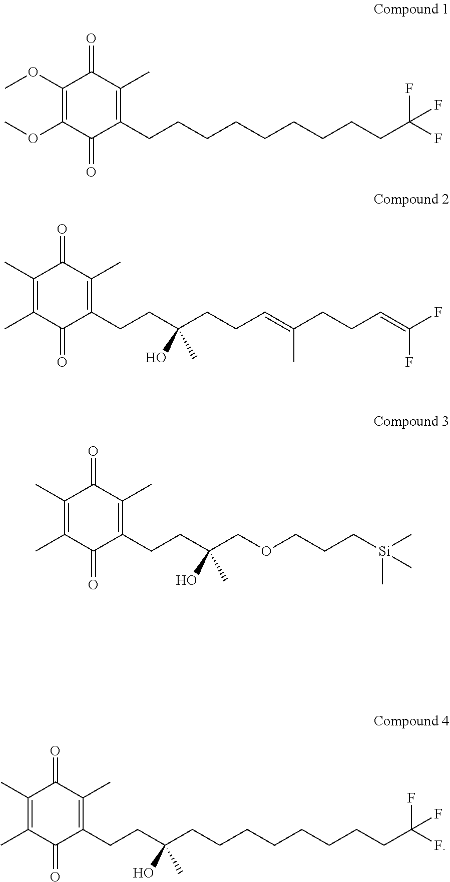

In some embodiments, the signs or symptoms of Leigh syndrome include difficulty swallowing, difficulty breathing, difficulty eating, hypotonia, lactic acidosis, developmental delays, cardiomyopathy, seizures, ataxia, dysarthria, dystonia, paralysis, impairment of respiratory and kidney function, color blindness and vision loss.

In some embodiments, the subject has been diagnosed with Leigh syndrome. In some embodiments, the subject is human.

In some embodiments, the Compound(s), composition, medicament, or formulation is/are administered daily for 6 weeks or more.

In some embodiments, the Compound(s), composition, medicament, or formulation is/are administered orally, topically, intranasally, systemically, intravenously, subcutaneously, intraperitoneally, intradermally, intraocularly, ophthalmically, intrathecally, intracerebroventricularly, iontophoretically, transmucosally, intravitreally, or intramuscularly.

In some embodiments, administration of the Compound(s), composition, medicament, or formulation, to the subject treats, prevents, ameliorates, inhibits, or delays the onset of a cardiomyopathy in the subject.

In some embodiments, the cardiomyopathy is hypertrophic cardiomyopathy, left ventricular hypertrophy, dilated cardiomyopathy, pericardial effusions and/or an arrythmia/conduction abnormality. In some embodiments, the cardiomyopathy is hypertrophic cardiomyopathy.

In some embodiments, administration of the Compound(s), composition, medicament, or formulation, to the subject lowers lactic acid levels in the subject's blood, urine, or cerebrospinal fluid (CSF).

In some embodiments, administration of the Compound(s), composition, medicament, or formulation, to the subject treats, prevents, ameliorates, inhibits, or delays the onset of multi-organ and/or central nervous system (CNS) damage, including lesions in the brainstem, basal ganglia, and spinal cord of the subject.

In some embodiments, administration of the Compound(s), composition, medicament, or formulation, to the subject treats, prevents, ameliorates, inhibits, or delays the onset of color blindness and/or vision loss of the subject.

In some embodiments, administration of the Compound(s), composition, medicament, or formulation, to the subject increases the lifespan of the subject.

In some embodiments, administration of the Compound(s), composition, medicament, or formulation, to the subject treats, prevents, ameliorates, inhibits, or delays the onset of seizures, ataxia, dysarthria, dystonia and/or paralysis of the subject.

### BRIEF DESCRIPTION OF THE DRAWINGS

FIG. 1 is a graphic illustration of data obtained for an in vitro assay designed to examine and quantify (i.e., by determining an EC₅₀) the ability of Compound 1 to exhibit Complex I by-pass activity.

FIG. 2 is a graphic illustration of data obtained for an in vitro assay designed to examine and quantify (i.e., by determining an EC₅₀) the ability of Compound 2 to exhibit Complex I by-pass activity.

FIG. 3 is a graphic illustration of data obtained for an in vitro assay designed to examine and quantify (i.e., by determining an EC₅₀) the ability of Compound 3 to exhibit Complex I by-pass activity.

FIG. 4 is a graphic illustration of data obtained for an in vitro assay designed to examine and quantify (i.e., by determining an EC₅₀) the ability of Compound 4 to exhibit Complex I by-pass activity.

FIG. 5 is a graphic illustration of data obtained for an in vitro assay designed to evaluate the impact of Compound 3 on ROS production generated by reverse electron transfer (RET) from Complex II to Complex I.

FIG. 6 is a graphic illustration of data obtained for an in vitro assay designed to evaluate the impact of Compound 4 on ROS production generated by reverse electron transfer (RET) from Complex II to Complex I.

FIG. 7 is a graphic illustration of data for an in vitro assay used to demonstrate the comparative effect of RSL3 on healthy verse diseased cells (i.e., fibroblasts derived from a patient with Leigh syndrome) for purposes of determining the concentration of RSL3 to be used in follow up studies with each cell line.

FIG. 8 is a graphic illustration of data for an in vitro assay used to examine and compare the ability of Compounds 1 to 4 and Vatiquinone to ameliorate RSL3 induced ferroptosis in patient derived fibroblasts from a healthy individual (cell line GM08402).

FIG. 9 is a graphic illustration of data for an in vitro assay used to examine and compare the ability of Compounds 1 to 4 and Vatiquinone to ameliorate RSL3 induced ferroptosis in patient derived fibroblasts from an individual diagnosed with Leigh syndrome (cell line GM03672).

FIG. 10 is a graphic illustration of data for an in vitro assay used to examine and compare the ability of Compounds 2 to 4 and Vatiquinone to act as a 15-lipoxygenase (15-LO) inhibitor in RSL3 induced ferroptosis in patient derived fibroblasts from a healthy individual.

FIG. 11 is a graphic illustration of data for an in vitro assay used to examine and compare the ability of Compounds 2 to 4 and Vatiquinone to act as a 15-lipoxygenase inhibitor in RSL3 induced ferroptosis in patient derived fibroblasts from an individual diagnosed with Leigh syndrome.

FIG. 12 is a graphic illustration of data for an in vitro assay used to examine and compare the ability of Compounds 1 to 4 and Vatiquinone to ameliorate the effects of injury to patient derived fibroblasts from a healthy individual caused by erastin.

FIG. 13 is a graphic illustration of data for an in vitro assay used to examine and compare the ability of Compounds 1 to 4 and Vatiquinone to ameliorate the effects of injury to patient derived fibroblasts from an individual diagnosed with Leigh syndrome caused by erastin.

FIG. 14 is a graphic illustration of data for an in vitro assay used to examine and compare the ability of Compounds 2, 3, 4 and Vatiquinone to act as a 15-lipoxygenase inhibitor in erastin injured fibroblasts from a healthy individual.

FIG. 15 is a graphic illustration of data for an in vitro assay used to examine and compare the ability of Compounds 1 to 4 and Vatiquinone to act as a 15-lipoxygenase inhibitor in erastin injured fibroblasts from an individual diagnosed with Leigh syndrome.

FIG. 16 is a graphic illustration of comparative data obtained in a mouse pharmacokinetic (PK) profile study for plasma where Compound 3 was studied at 20 mg/kg and 60 mg/kg SC dosing and Compound 4 was studied at 60 mg/kg SC dosing.

FIG. 17 is a graphic illustration of comparative data obtained in a PK profile study for plasma where Compound 3 was studied at 20 mg/kg SC dosing in mouse and 10 mg/kg SC dosing in rat (effectively 60 mg/m² regardless of species examined).

FIG. 18 is a graphic illustration of comparative data obtained in a PK profile study examining uptake of drug in mouse heart tissue resulting from subcutaneous administration of Compound 3 at 20 mg/kg and 60 mg/kg dosing and Compound 4 at 60 mg/kg dosing.

FIG. 19 is a graphic illustration of comparative data obtained in a PK profile study examining uptake of drug in mouse brain tissue resulting from subcutaneous administration of Compound 3 at 20 mg/kg and 60 mg/kg dosing and Compound 4 at 60 mg/kg dosing.

FIG. 20 is a bar graph presenting data obtained using a fibroblast line derived from a patient with Leigh Syndrome. It compares the membrane potential of untreated cells to that of cells treated with RSL3 alone and to that of cells treated with both RSL3 and Compound 3 (at 1 μM).

FIG. 21 is a bar graph presenting data obtained using a fibroblast line derived from a patient with Leigh Syndrome. It compares the membrane potential of untreated cells to that of cells treated with RSL3 alone and to that of cells treated with both RSL3 and Compound 4 (at 1 μM).

FIG. 22A is a chart showing mouse plasma PK profile after 5 days treatment with Compound 4 dosed at: (i) 60 mg/kg, 5% Kolliphor ELP (K-ELP) in PBS; (ii) 60 mg/kg, 15% K-ELP in PBS; and (iii) 180 mg/kg, 15% K-ELP in PBS.

FIGS. 22B-22C are charts showing mouse tissue (heart (FIG. 22B) and brain (FIG. 22C)) exposure profile after 5 days of treatment with Compound 4 dosed at: (i) 60 mg/kg, 5% Kolliphor ELP (K-ELP) in PBS; (ii) 60 mg/kg, 15% K-ELP in PBS; and (iii) 180 mg/kg, 15% K-ELP in PBS.

### DETAILED DESCRIPTION

It is to be appreciated that certain aspects, modes, embodiments, variations and features of the present disclosure are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

In practicing the present technology, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, N Y, 1987); and Meth. Enzymol., Vols. 154 and 155, Wu & Grossman, and Wu, Eds., respectively.

### I. Definitions

Definitions of specific functional groups and chemical terms are described in more detail below. The chemical elements are identified in accordance with the Periodic Table of the Elements, GAS version, Handbook of Chemistry and Physics, 7Sh Ed., inside cover. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

It is to be appreciated that certain aspects, modes, embodiments, variations and features of the technology are described below in various levels of detail in order to provide a substantial understanding of the present disclosure. The definitions of certain terms as used in this specification are provided below. Unless defined otherwise, all technical and scientific terms used herein generally have the same meaning as commonly understood by one of ordinary skill in the art to which this technology belongs.

As used in this specification and the appended embodiments, the singular forms “a”, “an” and “the” include plural references unless the content clearly dictates otherwise. For example, reference to “a cell” includes a combination of two or more cells, and the like.

As used herein, “administering” or the “administration” of an agent (i.e., a therapeutic agent) or compound/drug product (including a composition (i.e., a formulation or medicament)) to a subject includes any route of introducing or delivering to a subject a therapeutic agent/compound/drug product to perform its intended function. Administration may be carried out by any suitable route, such as oral administration. Administration can be carried out subcutaneously. Administration can be carried out intravenously. Administration can be carried out intraocularly. Administration can be carried out systemically. Alternatively, administration may be carried out topically, intranasally, intraperitoneally, intradermally, ophthalmically, intrathecally, intracerebroventricularly, iontophoretically, transmucosally, intravitreally, or intramuscularly. Administration includes self-administration, the administration by another or administration by use of a device (e.g., an infusion pump).

As used herein, to “ameliorate” or “ameliorating” a disease, disorder or condition refers to results that, in a statistical sample or specific subject, make the occurrence of the disease, disorder or condition (or a sign or symptom thereof) better or more tolerable in a sample or subject administered a therapeutic agent relative to a control sample, control subject or group of control subjects.

As used herein the terms “carrier” or “pharmaceutically acceptable carrier” refer to a diluent, adjuvant, excipient, or vehicle with which a therapeutic agent/compound/drug product/composition (including a formulation or medicament) is administered or formulated for administration. Non-limiting examples of such pharmaceutically acceptable carriers include liquids, such as water, saline, oils and solids, such as gum acacia, gelatin, starch paste, talc, keratin, colloidal silica, silica particles (nanoparticles or microparticles) urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

As used herein, the phrase “delaying the onset of” refers to, in a statistical sample, postponing, hindering the occurrence of a disease, disorder or condition, or causing one or more signs, or symptoms of a disease, disorder or condition to occur more slowly than normal, in a sample or subject administered a therapeutic agent or agents relative to a control sample, control subject or group of control subjects.

As used herein, the term “effective amount” refers to a quantity of a therapeutic agent/compound/composition/drug product sufficient to achieve a desired therapeutic and/or prophylactic effect, e.g., an amount that treats, prevents, inhibits, ameliorates, or delays the onset of a disease, disorder or condition, or the physiological signs or symptoms of the disease, disorder or condition. In the context of therapeutic or prophylactic applications, in some embodiments, the amount of a therapeutic agent/compound/composition/drug product administered to the subject will depend on the type and severity of the disease and on the characteristics of the individual, such as general health, age, sex, body weight and tolerance to drugs. In some embodiments, it will also depend on the degree, severity and type of disease. The skilled artisan will be able to determine appropriate dosages depending on these and other factors. The compounds/compositions/drug products can also be administered in combination with one or more additional therapeutic compounds/agents (a so called “co-administration” where, for example, the additional or other therapeutic agent(s) could be administered simultaneously, sequentially or by separate administration).

As used herein, the term “hydrate” refers to a compound which is associated (e.g., complexed) with water. The number of the water molecules contained in a hydrate of a compound may be (or may not be) in a definite ratio to the number of the compound molecules in the hydrate.

As used herein, “inhibit” or “inhibiting” refers to the reduction in a sign, symptom, or condition (e.g., risk factor) associated with Leigh syndrome. In one embodiment, inhibit or inhibiting refers to the reduction by at least a statistically significant amount compared to a control, control subject or group of control subjects. In one embodiment, inhibit or inhibiting refers to a reduction by at least 5 percent compared to control, control subject or group of control subjects. In various individual embodiments, inhibit or inhibiting refers to a reduction by at least 1, 2, 3, 4, 5, 10, 15, 20, 25, 30, 33, 40, 50, 60, 67, 70, 75, 80, 90, 95, or 99 percent compared to a control, control subject, or group of control subjects.

As used herein, the term “pharmaceutically acceptable salt” refers to a salt of a therapeutic compound that can be prepared with relatively nontoxic acids or bases, depending on the particular substituents found on the compounds described herein. When compounds contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, magnesium salt, or a similar salt. When compounds contain relatively basic functionalities, acid addition salts can be obtained by contacting the neutral form of such compounds with a sufficient amount of the desired acid, either neat or in a suitable inert solvent. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-methylmorpholine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine (NEt₃), trimethylamine, tripropylamine, tromethamine and the like, such as where the salt includes the protonated form of the organic base (e.g., [HNEt₃]+). Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphorsulfonic, edisylic, ethanesulfonic, isethionic, methanesulfonic, naphthalenesulfonic, naphthalene-1,5-disulfonic, naphthalene-2,6-disulfonic, p-toluenesulfonic acids (PTSA)), xinafoic acid, and the like. In some embodiments, the pharmaceutically acceptable counterion is selected from the group consisting of acetate, benzoate, besylate, bromide, camphorsulfonate, chloride, chlorotheophyllinate, citrate, ethanedisulfonate, fumarate, gluceptate, gluconate, glucoronate, hippurate, iodide, isethionate, lactate, lactobionate, laurylsulfate, malate, maleate, mesylate, methylsulfate, naphthoate, sapsylate, nitrate, octadecanoate, oleate, oxalate, pamoate, phosphate, polygalacturonate, succinate, sulfate, sulfosalicylate, tartrate, tosylate, and trifluoroacetate. In some embodiments, the salt is a tartrate salt, a fumarate salt, a citrate salt, a benzoate salt, a succinate salt, a suberate salt, a lactate salt, an oxalate salt, a phthalate salt, a methanesulfonate salt, a benzenesulfonate salt, a maleate salt, a trifluoroacetate salt, a hydrochloride salt, or a tosylate salt. Also included are salts of amino acids such as arginate and the like, and salts of organic acids such as glucuronic or galactunoric acids and the like (see, e.g., Berge et al, Journal of Pharmaceutical Science 66:1-19 (1977)). Certain specific compounds may contain both basic and acidic functionalities that allow the compounds to be converted into either base or acid addition salts or exist in zwitterionic form. These salts may be prepared by methods known to those skilled in the art. Any other pharmaceutically acceptable carriers known to those of skill in the art are suitable for use with the present technology.

As used herein, “prevention” or “preventing” of a disease, disorder, or condition refers to results that, in a statistical sample, exhibit a reduction in the occurrence of the disease, disorder, or condition in a sample or subject administered a therapeutic agent or agents relative to a control sample, control subject or group of control subjects. Such prevention is sometimes referred to as a prophylactic treatment.

As used herein, the term “separate” refers to an administration of at least two active ingredients (e.g., therapeutic agents) at the same time or at substantially the same time by different routes.

As used herein, the term “sequential” refers to administration of at least two active ingredients (e.g., therapeutic agents) at different times, the administration route being identical or different. More particularly, sequential administration refers to the whole administration of one of the active ingredients (e.g., therapeutic agents) before administration of the other or others commences. It is thus possible to administer one of the active ingredients over several minutes, hours, or days before administering the other active ingredient or ingredients. There is no simultaneous treatment in this definition.

As used herein, the term “simultaneous” refers to the administration of at least two active ingredients (e.g., therapeutic agents) by the same route and at the same time or at substantially the same time.

As used herein, the term “solvate” refers to forms of the compound that are associated with a solvent, possibly by a solvolysis reaction. This physical association may include hydrogen bonding. Conventional solvents include water, methanol, ethanol, isopropanol, acetic acid, ethyl acetate, acetone, hexane(s), dimethyl sulfoxide (DMSO), tetrahydrofuran (THF), diethyl ether, and the like.

As used herein, a “subject” refers to a living animal. In various embodiments, a subject is a mammal. In various embodiments, a subject is a non-human mammal, including, without limitation, a mouse, rat, hamster, guinea pig, rabbit, sheep, goat, cat, dog, pig, minipig, horse, cow, or non-human primate. In certain embodiments, the subject is a human.

It is also to be appreciated that the various modes of treatment or prevention of medical conditions as described herein, in some embodiments, are intended to mean “substantial,” which includes total but also less than total treatment, prevention, amelioration, or inhibition and wherein some biologically or medically relevant result is achieved.

As used herein, a “synergistic therapeutic effect” refers to a greater-than-additive therapeutic effect which is produced by a combination of at least two agents, and which exceeds that which would otherwise result from the individual administration of the agents.

As used herein, the term “tautomer” refers to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

As used herein, the terms “treating” or “treatment” refer to therapeutic treatment, wherein the object is to reduce, alleviate or slow down (lessen) a pre-existing disease, disorder or condition, or its related signs, or symptoms. By way of example, but not by way of limitation, a subject is successfully “treated” for a disease if, after receiving an effective amount of the therapeutic agent/compound/composition/drug product or a pharmaceutically acceptable salt, stereoisomer, tautomer, hydrate, and/or solvate thereof, the subject shows observable and/or measurable reduction in or absence of one or more signs, symptoms or conditions associated with the disease or disorder. It is also to be appreciated that the various modes of treatment of medical conditions as described are intended to mean “substantial,” which includes total alleviation of signs or symptoms of the disease disorder, or condition as well as “partial,” where some biologically or medically relevant result is achieved.

### II. Pharmaceutical Compositions, Routes of Administration, and Dosing

The methods, uses and compositions of the present application utilize a therapeutically effective amount of Compound 1, Compound 2, Compound 3, Compound 4, or a mixture of any two or more of Compounds 1 to 4, or pharmaceutically acceptable salts, stereoisomers, mixtures of stereoisomers, tautomers, hydrates, and/or solvates thereof, wherein Compounds 1 to 4 have the following structures:

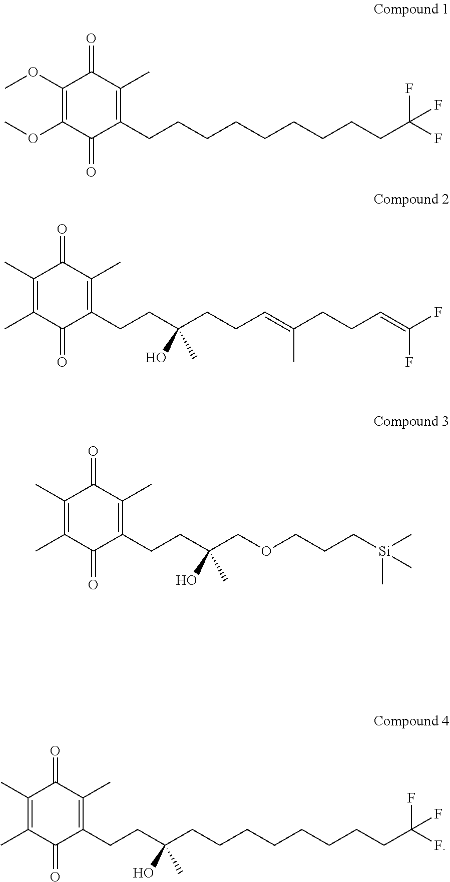

Compounds 1 to 4, or a mixture of any two or more of Compounds 1 to 4, or pharmaceutically acceptable salts, stereoisomers, mixtures of stereoisomers, tautomers, hydrates, and/or solvates thereof can be formulated into a drug product suitable for administration to a subject in need thereof. Such drug product can be referred to as a composition, formulation or medicament depending on its usage. Any mixture prepared by mixing any one or more of Compounds 1 to 4, or pharmaceutically acceptable salts, stereoisomers, mixtures of stereoisomers, tautomers, hydrates, and/or solvates thereof with a solvent and/or other compound(s) is a composition or formulation and may or may not be intended for administration to a subject. A medicament is generally considered a composition or formulation specifically prepared for administration to a subject to address a disease, disorder, or condition (e.g., Leigh syndrome). For purposes of brevity, whenever there is a reference to “Compounds 1 to 4 or mixtures of two more of Compounds 1 to 4” herein, this reference is intended to implicitly include any or pharmaceutically acceptable salts, stereoisomers, mixtures of stereoisomers, tautomers, hydrates, and/or solvates of Compounds 1 to 4.

Compounds 1 to 4, or a mixture of any two or more of Compounds 1 to 4 disclosed herein can be used, alone or in combination, with other therapeutic agent(s) to address the needs of subjects suffering from Leigh syndrome. In order to be administered to a subject in need thereof, Compounds 1 to 4, or a mixture of any two or more of Compounds 1 to 4 will generally need to be formulated (for individual administration or in a combined formulation) for the intended route of administration. In some embodiments, the same route of administration can be used to deliver Compounds 1 to 4, or a mixture of any two or more of Compounds 1 to 4. In some embodiments, two or more of Compounds 1 to 4, or a mixture of any two or more of Compounds 1 to 4 can be administered by different routes. The formulated product(s) can be considered a composition or medicament comprising Compounds 1 to 4, or a mixture of any two or more of Compounds 1 to 4 and optionally one or more other (i.e., additional) therapeutic agents.

In some embodiments, the therapeutic agent(s) can be formulated with little or no excipient or carrier. In some embodiments, the therapeutic agent(s) can be formulated such that the majority of the formulation is excipient or carrier. In brief, one of skill in the art will tailor the formulation to have a suitable amount of excipient or carrier based on the needs/condition of the subject, the kind and extent of the disease to be treated; the properties of the therapeutic agent or agents to be delivered and the selected mode of administration of the particular therapeutic agent or agents.

In certain embodiments, a pharmaceutical composition may further comprise at least one therapeutic agent other than Compounds 1 to 4, or a mixture of any two or more of Compounds 1 to 4 (e.g. another (or additional) therapeutic agent for use in combination with the Compounds 1 to 4, or a mixture of any two or more of Compounds 1 to 4). The at least one other/additional therapeutic agent can be an agent useful in the treatment of Leigh syndrome or could, for example, be administered to ameliorate the side effect of the administration of Compounds 1 to 4, or a mixture of any two or more of Compounds 1 to 4 (e.g., to address a side reaction or because the combination therapy is preferred for the treatment of Leigh syndrome). Thus, in some embodiments, pharmaceutical compositions can be prepared, for example, by combining Compounds 1 to 4, or a mixture of any two or more of Compounds 1 to 4 with a pharmaceutically acceptable carrier and, optionally, one or more additional therapeutical agents or otherwise merely administering the other/additional therapeutic agent(s) in combination with the administration of Compounds 1 to 4, or a mixture of any two or more of Compounds 1 to 4.

Pharmaceutical compositions may contain an effective amount of one or more of the therapeutic agent or agents as described herein and may optionally be disbursed (e.g. dissolved, suspended or otherwise) in a pharmaceutically acceptable carrier. The components of the pharmaceutical composition(s) may also be capable of being commingled with the compounds of the present application, and with each other, in a manner such that there is no interaction that would substantially impair the desired pharmaceutical efficiency.

As stated above, an “effective amount” refers to any amount of a particular therapeutic agent that is sufficient to achieve a desired biological effect. Combined with the teachings provided herein, by choosing among the various therapeutic compound(s) and weighing factors such as potency, relative bioavailability, patient body weight, severity of adverse side-effects and mode of administration, an effective prophylactic (i.e. preventative) or therapeutic treatment regimen can be planned which does not cause substantial unwanted toxicity and yet is effective to address the particular condition, disorder or disease of a particular subject in a therapeutic way. The effective amount of a therapeutic agent for any particular indication can vary depending on such factors as the disease, disorder or condition being treated, the particular compound or compounds being administered, the size of the subject, the age of the subject, the overall health of the subject and/or the severity of the disease, disorder or condition. The effective amount may be determined during pre-clinical trials and/or clinical trials by methods familiar to physicians and clinicians. One of ordinary skill in the art can empirically determine the effective amount of a particular therapeutic agent or agents without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein. A dose may be administered by oneself, by another or by way of a device (e.g., a pump).

For any therapeutic compound described herein the therapeutically effective amount can, for example, be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

Therapeutic compounds (alone or as formulated in a pharmaceutical composition/medicament) for use in therapy or prevention can be tested in suitable animal model systems. Suitable animal model systems include, but are not limited to, rats, mice, chicken, cows, monkeys, rabbits, pigs, minipigs and the like, prior to testing in human subjects. In vivo testing of any animal model system known in the art can be used prior to administration to human subjects. In some embodiments, dosing can be tested directly in humans.

Dosage, toxicity and therapeutic efficacy of any therapeutic agents or compositions (e.g., formulations or medicaments comprising Compounds 1 to 4, or a mixture of any two or more of Compounds 1 to 4), other/additional therapeutic agents, or mixtures thereof can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, in such cases it may be prudent to design a delivery system that targets such compounds to the site of affected tissue in order to minimize potential damage to uninfected cells and, thereby, reduce side effects.

An exemplary treatment regime can, for example, entail administration once per day, twice per day, thrice per day, once a week, or once a month. In therapeutic applications, a relatively high dosage at relatively short intervals is sometimes required until progression of the disease is delayed, reduced or terminated, or until the subject shows partial or complete amelioration of symptoms of disease. Thereafter, the patient can be administered a prophylactic regimen.

For use in therapy, an effective amount of the therapeutic compound (alone or as formulated) can be administered to a subject by any mode that delivers the compound to the desired surface. Administering a pharmaceutical composition may be accomplished by any means known to the skilled artisan. Routes of administration include but are not limited to oral, topical, intranasal, systemic, intravenous, subcutaneous, intraperitoneal, intradermal, intraocular, ophthalmical, intrathecal, intracerebroventricular, iontophoretical, transmucosal, intravitreal, or intramuscular administration. Administration includes self-administration, administration by another and administration by a device (e.g., a pump).

A therapeutic compound/agent disclosed herein (e.g., Compounds 1 to 4, or a mixture of any two or more of Compounds 1 to 4) can be delivered to the subject in a formulation or medicament (i.e., a pharmaceutical composition). Formulations and medicaments can be prepared by, for example, dissolving or suspending a therapeutic compound/agent disclosed herein (e.g., Compounds 1 to 4, or a mixture of any two or more of Compounds 1 to 4) in water, a solvent, a pharmaceutically acceptable carrier, salt, (e.g., NaCl or sodium phosphate), buffering agents, preservatives, compatible carriers, adjuvants, and optionally other therapeutically acceptable ingredients.

The pharmaceutical compositions (e.g. a formulation or medicament) can include a carrier (e.g., a pharmaceutically acceptable carrier), which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars (e.g., trehalose), polyalcohols such as mannitol, sorbitol, or sodium chloride in the composition. Prolonged absorption of the injectable compositions can be brought about by including in the composition an agent that delays absorption, for example, aluminum monostearate or gelatin.

Solutions or suspensions (e.g., a formulation or medicament) used for parenteral, intradermal, subcutaneous or intraocular application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided alone or in a kit containing all necessary equipment (e.g., vials of drug, vials of diluent, syringes and needles) for a treatment course (e.g., 1, 2, 3, 4, 5, 6, 7 days or more of treatment).

The therapeutic compounds/agents or pharmaceutical compositions, when it is desirable to deliver them systemically, may be formulated for parenteral administration by injection, e.g., by bolus injection or continuous infusion (for example by IV injection or via a pump to meter the administration over a defined time). Formulations for injection may be presented in unit dosage form, e.g., in ampoules or in multi-dose containers, with an added preservative. Pharmaceutical compositions may take such forms as suspensions, solutions or emulsions in oily or aqueous vehicles, and may contain formulatory agents such as suspending, stabilizing and/or dispersing agents. Additionally, suspensions of the therapeutic compounds (e.g., Compounds 1 to 4, or a mixture of any two or more of Compounds 1 to 4) may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Systemic formulations include those designed for administration by injection, e.g., subcutaneous, intravenous, intramuscular, intrathecal, or intraperitoneal injection, as well as those designed for transdermal, transmucosal oral or pulmonary administration.

For intravenous and other parenteral routes of administration, a compound (e.g., Compounds 1 to 4, or a mixture of any two or more of Compounds 1 to 4) can be formulated as a lyophilized preparation, as a lyophilized preparation of liposome-intercalated or lipid-encapsulated therapeutic compound(s), as a lipid complex in aqueous suspension, or as a salt complex. Lyophilized formulations are generally reconstituted in suitable aqueous solution, e.g., in sterile water or saline, shortly prior to administration.

Pharmaceutical compositions (e.g., a formulation or medicament) suitable for injection can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). A composition for administration by injection will generally be sterile and should be fluid to the extent that easy syringability exists. It should be stable under the conditions of manufacture and storage and may be preserved against the contaminating action of microorganisms such as bacteria and fungi.

Sterile injectable solutions (e.g., a formulation or medicament) can be prepared by incorporating the therapeutic compound(s) (e.g., Compounds 1 to 4, or a mixture of any two or more of Compounds 1 to 4) in the required amount in an appropriate solvent with one or a combination of ingredients enumerated above, as required, followed by filtered sterilization. Generally, dispersions are prepared by incorporating the therapeutic compound(s) into a sterile vehicle, that contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

For oral administration, the compounds can be formulated readily by combining the therapeutic compound(s) (e.g., Compounds 1 to 4, or a mixture of any two or more of Compounds 1 to 4) with pharmaceutically acceptable carriers well known in the art. Such carriers enable the therapeutic compound(s) to be formulated as tablets, pills, dragees, capsules, liquids, gels, syrups, slurries, suspensions and the like, for oral ingestion by a subject to be treated. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel®, or corn starch; a lubricant such as magnesium stearate or sterates; a glidant such as colloidal silicon dioxide; a sweetening agent such as sucrose or saccharin; or a flavoring agent such as peppermint, methyl salicylate, or orange flavoring.

Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above that may be chemically modified so that oral delivery of the derivative is efficacious. Generally, the chemical modification contemplated is the attachment of at least one moiety to the therapeutic agent(s), ingredient(s), and/or excipient(s), where said moiety permits (a) inhibition of acid hydrolysis; and (b) uptake into the blood stream from the stomach or intestine. Also desired is the increase in overall stability of the therapeutic agent(s), ingredient(s), and/or excipient(s) and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol (PEG) moieties of various molecular weights are suitable.

For the formulation of the therapeutic agent(s), ingredient(s), and/or excipient(s), the location of release may be the stomach, the small intestine (the duodenum, the jejunum, or the ileum), or the large intestine. One skilled in the art has available formulations which will not dissolve in the stomach yet will release the material in the duodenum or elsewhere in the intestine. Preferably, the release will avoid the deleterious effects of the stomach environment, either by protection of a therapeutic compound/agent or by release of the biologically active material beyond the stomach environment, such as in the intestine.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic compound(s)/agent(s) (which term “therapeutic compound(s)/agent(s)” as used herein is intended to refer to Compounds 1 to 4, or a mixture of any two or more of Compounds 1 to 4 and any other active pharmaceutical ingredient (e.g., other therapeutic agent) that can be administered in a combination with Compounds 1 to 4, or a mixture of any two or more of Compounds 1 to 4. The formulation can comprise fine multi-particulates in the form of granules or pellets of particle size about 1-2 mm. The formulation of the material for capsule administration could also be as a powder, lightly compressed plugs or even as tablets. The therapeutic compound(s)/agent(s) or pharmaceutical composition(s) could be prepared by compression.

Colorants and flavoring agents may all be included. For example, the therapeutic compound(s)/agent(s) or pharmaceutical composition(s) may be formulated and then further contained within an edible product, such as a refrigerated beverage containing colorants and flavoring agents.

One may dilute or increase the volume of the therapeutic compound(s)/agent(s) or pharmaceutical composition(s) with an inert material. These diluents could include carbohydrates, especially mannitol, lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo®, Emdex®, STARCH 1500®, Emcompress® and Avicel®.

Disintegrants may be included in the formulation of the therapeutic compound(s)/agent(s) or pharmaceutical composition(s) into a solid dosage form. Materials used as disintegrates include but are not limited to starch, including the commercial disintegrant based on starch, Explotab. Sodium starch glycolate, Amberlite®, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, karaya gum or tragacanth. Alginic acid and its sodium salt are also useful as disintegrants.

Binders may be used to hold the therapeutic agent(s) together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic agent(s).

An anti-frictional agent may be included in the formulation of the therapeutic compound(s)/agent(s) or pharmaceutical composition(s) to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol (PEG) of various molecular weights, Carbowax™ 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic compound(s)/agent(s) or pharmaceutical composition(s) into the aqueous environment, a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation or medicament as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation or medicament disclosed herein or derivative either alone or as a mixture in different ratios.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the therapeutic compound(s) may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the therapeutic compound(s)/agent(s) or pharmaceutical composition(s) may be formulated as solutions, gels, ointments, creams, suspensions, etc. as are well-known in the art. Solutions, gels, ointments, creams or suspensions may be administered topically. The compounds may also be formulated in rectal or vaginal compositions such as suppositories or retention enemas, e.g., containing conventional suppository bases such as cocoa butter or other glycerides.

For administration by inhalation, therapeutic compound(s)/agent(s) or pharmaceutical composition(s) for use according to the present application may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In some embodiments, the formulation, medicament and/or therapeutic compound(s)/agent(s) can be delivered in the form of an aerosol spray from a pressurized container or dispenser, which contains a suitable propellant, e.g., a gas such as carbon dioxide, or a nebulizer. Such methods include those described in U.S. Pat. No. 6,468,798. In the case of a pressurized aerosol the dosage unit may be determined by providing a valve to deliver a metered amount. In the case of a pressurized aerosol the dosage unit may be determined by providing a valve to deliver a metered amount. For example, capsules and cartridges of e.g., gelatin for use in an inhaler or insufflator may be formulated containing a powder mix of the therapeutic compound/agent and a suitable powder base such as lactose or starch. Alternatively, the therapeutic compound(s)/agent(s) or pharmaceutical composition(s) may be in powder form for constitution with a suitable vehicle, e.g., sterile pyrogen-free water, before use.

Nasal delivery of a therapeutic compound(s)/agent(s) or pharmaceutical composition(s) is also contemplated. Nasal delivery allows the passage of therapeutic compound(s)/agent(s) or pharmaceutical composition(s) to the blood stream directly after administering the therapeutic compound(s)/agent(s) or pharmaceutical composition(s) to the nose, without the necessity for deposition of the product in the lung. Formulations for nasal delivery include those with dextran or cyclodextran.

For nasal administration, one type of useful device is a small, hard bottle to which a metered dose sprayer is attached. In some embodiments, the metered dose is delivered by drawing a pharmaceutical composition (in solution form) into a chamber of defined volume, which chamber has an aperture dimensioned to aerosolize and aerosol formulation by forming a spray when a liquid in the chamber is compressed. The chamber is compressed to administer the therapeutic compound(s)/agent(s) or pharmaceutical composition(s). In a specific embodiment, the chamber is a piston arrangement. Such devices are commercially available.

Alternatively, a plastic squeeze bottle with an aperture or opening dimensioned to aerosolize an aerosol formulation by forming a spray when squeezed can be used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the therapeutic compound(s)/agent(s) or pharmaceutical composition(s).

Also contemplated herein is pulmonary delivery of the compounds disclosed herein. The therapeutic compound(s)/agent(s) or pharmaceutical composition(s) is/are delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13 (suppl. 5): 143-146 (1989) (endothelin-1); Hubbard et al., Annal Int Med 3:206-212 (1989) (α1-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995, to Wong et al.

Contemplated for use in the practice of this technology are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this technology are the Ultravent™ nebulizer, manufactured by Mallinckrodt, Inc., St. Louis, Mo.; the Acorn II® nebulizer, manufactured by Marquest Medical Products, Englewood, Colo.; the Ventolin® metered dose inhaler, manufactured by Glaxo Inc., Research Triangle Park, North Carolina; and the Spinhaler® powder inhaler, manufactured by Fisons Corp., Bedford, Mass.

All such devices require the use of formulations suitable for the dispensing of the therapeutic compound(s)/agent(s) or pharmaceutical composition(s). Typically, each formulation is specific to the type of device employed and may involve the use of an appropriate propellant material, in addition to the usual diluents, adjuvants and/or carriers useful in therapy. Also, the use of liposomes, microcapsules, microspheres, nanoparticles, nanospheres, inclusion complexes, or other types of carriers is contemplated.

Formulations suitable for use with a nebulizer, either jet or ultrasonic, can, for example, comprise therapeutic compound(s)/agent(s) or pharmaceutical composition(s) dissolved in water at a concentration of about 0.01 to 50 mg of biologically active compound per mL of solution. The formulation may also include a buffer and optionally a simple sugar (e.g., for inhibitor stabilization and regulation of osmotic pressure). The nebulizer formulation may also contain a surfactant, to reduce or prevent surface induced aggregation of the therapeutic compound(s)/agent(s) or pharmaceutical composition(s) disclosed herein caused by atomization of the solution in forming the aerosol.

Formulations for use with a metered-dose inhaler device may generally comprise a finely divided powder comprising the therapeutic compound(s)/agent(s) or pharmaceutical composition(s) disclosed herein suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device may comprise a finely divided dry powder containing the therapeutic compound(s)/agent(s) or pharmaceutical composition(s) and may also include a bulking agent, such as lactose, sorbitol, sucrose, or mannitol in amounts which facilitate dispersal of the powder from the device, e.g., 50 to 90% by weight of the formulation. The compound(s)/therapeutic agent(s)/pharmaceutical composition(s) can advantageously be prepared in particulate or nanoparticulate form with an average particle size of less than 10 micrometers (μm), most preferably 0.5 to 5 μm, for most effective delivery to the deep lung.

For ophthalmic or intraocular indications, any suitable mode of delivering the therapeutic compound(s)/agent(s) or pharmaceutical composition(s) to the eye or regions near the eye can be used. For ophthalmic formulations generally, see Mitra (ed.), Ophthalmic Drug Delivery Systems, Marcel Dekker, Inc., New York, N.Y. (1993) and also Havener, W. H., Ocular Pharmacology, C. V. Mosby Co., St. Louis (1983). Nonlimiting examples of pharmaceutical compositions suitable for administration in or near the eye include, but are not limited to, ocular inserts, minitablets, and topical formulations such as eye drops, ointments, and in situ gels. In one embodiment, a contact lens is coated with a pharmaceutical composition (or contains a pharmaceutical composition encapsulated therein) comprising a therapeutic compound/agent. In some embodiments, a single dose can comprise from between 0.1 ng to 5000 μg, 1 ng to 500 μg, or 10 ng to 100 μg of the therapeutic compound(s)/agent(s) or pharmaceutical composition(s) administered to the eye.

Eye drops can comprise a sterile liquid formulation that can be administered directly to the eye. In some embodiments, eye drops comprise at least one therapeutic agent (and possibly several) and may further comprise one or more preservatives. In some embodiments, the optimum pH for eye drops equals that of tear fluid and is about 7.4, the pH may be within any range that is not harmful to the eye of the subject. For eye drops, the therapeutic compound(s)/agent(s) can be present in the drop solution from about 0.1% to about 5% (w/v or v/v depending on the physical nature (i.e. solid or liquid) of the active ingredient). In some embodiments, the therapeutic compound/agent can be present in the drop solution from about 1% to about 3% (w/v or v/v, as appropriate). In some embodiments, the therapeutic compound/agent can be present in the drop solution from about 0.2% to about 1.5% (w/v or v/v, as appropriate). In some embodiments, the therapeutic compound/agent can be present in the drop solution from about 0.1% to about 1.0% (w/v or v/v, as appropriate).

In situ gels are viscous liquids, showing the ability to undergo sol-to-gel transitions when influenced by external factors, such as appropriate pH, temperature, pressure and/or the presence of electrolytes. This property causes slowing of drug drainage from the eyeball surface and increase of the active ingredient bioavailability. Polymers commonly used in in situ gel formulations include, but are not limited to, gellan gum, poloxamer, silicone containing formulations, silica-based formulations, and cellulose acetate phthalate. In some embodiments, the therapeutic compound(s)/agent(s) or pharmaceutical composition(s) is/are formulated into an in-situ gel (as the formulation/medicament).

For topical ophthalmic administration, therapeutic compound(s)/agent(s) or pharmaceutical composition(s) is/are may be formulated as solutions, gels, ointments, creams, suspensions, etc. as are well-known in the art. Ointments are semisolid dosage forms for external use such as topical use for the eye or skin. In some embodiments, ointments comprise a solid or semisolid hydrocarbon base of melting or softening point close to human core temperature. In some embodiments, an ointment applied to the eye decomposes into small drops, which stay for a longer time period in conjunctival sac, thus increasing bioavailability.

Ocular inserts are solid or semisolid dosage forms without disadvantages of traditional ophthalmic drug forms. They are less susceptible to defense mechanisms like outflow through nasolacrimal duct, show the ability to stay in conjunctival sac for a longer period, and can be more stable than conventional dosage forms. They also offer advantages such as accurate dosing of one or more therapeutic compound(s)/agent(s) or pharmaceutical composition(s), slow release of one or more therapeutic compound(s)/agent(s) with constant speed and limiting of one or more therapeutic compounds'/agents' systemic absorption. In some embodiments, an ocular insert comprises one or more therapeutic compound(s)/agent(s) and one or more polymeric materials. The polymeric materials can include, but are not limited to, methylcellulose and its derivatives (e.g., hydroxypropyl methylcellulose (HPMC)), ethylcellulose, polyvinylpyrrolidone (PVP K-90), polyvinyl alcohol, chitosan, carboxymethyl chitosan, gelatin, and various mixtures of the aforementioned polymers. An ocular insert can comprise silica. An ocular insert can comprise liposomes, nanoparticles or microparticles of degradable or biodegradable polymer (as described in more detail below).

Minitablets are biodegradable, solid drug forms, that transit into gels after application to the conjunctival sac, thereby extending the period of contact between active ingredient (i.e., the therapeutic compound(s)/agent(s)) and the eyeball surface, which in turn increases a therapeutic compounds'/agents' bioavailability. The advantages of minitablets include easy application to conjunctival sac, resistance to defense mechanisms like tearing or outflow through nasolacrimal duct, longer contact with the cornea caused by presence of mucoadhesive polymers, and gradual release of the active ingredient from the formulation in the place of application due to the swelling of the outer carrier layers. Minitablets can comprise one or more therapeutic compound(s)/agent(s) and one or more polymers. Nonlimiting examples of polymers suitable for use in in a minitablet formulation include cellulose derivatives, like hydroxypropyl methylcellulose (HPMC), hydroxyethyl cellulose (HEC), sodium carboxymethyl cellulose, ethyl cellulose, acrylates (e.g., polyacrylic acid and its cross-linked forms), Carbopol® or carbomer, chitosan, and starch (e.g., drum-dried waxy maize starch). In some embodiments, minitablets further comprise one or more excipients. Nonlimiting examples of excipients include mannitol and magnesium stearate.

The ophthalmic or intraocular formulations and medicaments may contain non-toxic auxiliary substances such as antibacterial components which are generally non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid (EDTA), and the like.

In some embodiments, the viscosity of the ocular formulation comprising one or more therapeutic compound(s)/agent(s) is increased to improve contact with the cornea and bioavailability in the eye. Viscosity can be increased by the addition of hydrophilic polymers of high molecular weight which do not diffuse through biological membranes and which form three-dimensional networks in the water. Nonlimiting examples of such polymers include polyvinyl alcohol, poloxamers, hyaluronic acid, carbomers, and polysaccharides, cellulose derivatives, gellan gum, and xanthan gum.

In addition to the formulations described above, therapeutic compound(s)/agent(s) or pharmaceutical composition(s) may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

In some embodiments, the therapeutic compound(s)/agent(s) or pharmaceutical composition(s) is/are administered as a depot formulation wherein the active therapeutic agent(s) is/are encapsulated by, or disposed within, silica-based microparticles. Such a formulation may be a controlled-release, delayed-release or extended release formulation (terms are defined below). Such controlled-release, delayed release or extended release formulation may comprise particles, such as microparticles or nanoparticles.

The pharmaceutical compositions also may comprise suitable solid or gel-phase carriers or excipients. Examples of such carriers or excipients include but are not limited to calcium carbonate, calcium phosphate, various sugars, starches, cellulose derivatives, gelatin, silica/silicone and polymers such as polyethylene glycols.

Suitable liquid or solid pharmaceutical preparation forms (e.g., a formulation or medicament) can, for example, be aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions/formulations may also include granules, powders, tablets, coated tablets, (micro) capsules, suppositories, syrups, emulsions, suspensions, creams, drops or preparations with protracted release of therapeutic compound(s), in whose preparation excipients and additives and/or auxiliaries such as disintegrants, binders, coating agents, swelling agents, lubricants, flavorings, sweeteners or solubilizers are customarily used as described above. The pharmaceutical compositions can be suitable for use in a variety of drug delivery systems. For a brief review of methods for drug delivery, see Langer R, Science 249:1527-33 (1990).

The therapeutic compound(s)/agent(s) or pharmaceutical composition(s) may be provided in particles. Particles as used herein means nanoparticles or microparticles (or in some instances larger particles) which can consist in whole or in part of the therapeutic compound(s)/agent(s) as described herein. The particles may contain the therapeutic compound(s)/agent(s) in a core surrounded by a coating, including, but not limited to, an enteric coating. The therapeutic compound(s)/agent(s) also may be dispersed throughout the particles. The therapeutic compound(s)/agent(s) also may be adsorbed into the particles. The particles may be of any order release kinetics, including zero-order release, first-order release, second-order release, delayed release, sustained release, immediate release, and any combination thereof, etc. The particle may include, in addition to any therapeutic compound(s)/agent(s), any of those materials routinely used in the art of pharmacy and medicine, including, but not limited to, erodible, non-erodible, biodegradable, or nonbiodegradable material or combinations thereof. The particles may be microcapsules which contain the therapeutic compound(s)/agent(s) in a solution or in a semi-solid state. The particles may be of virtually any shape.

Both non-biodegradable and biodegradable polymeric materials can be used in the manufacture of particles for delivering the therapeutic compound(s)/agent(s). Such polymers may be natural or synthetic polymers. The polymer can be selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, polyethylene glycols (PEGs), polyvinylalcohols (PVAs), poly(methyl methacrylates), poly(ethyl methacrylates), poly(butylmethacrylate), poly(isobutyl methacrylate), poly-lactic acid (PLA), poly(lactic-co-glycolic) acid (PLGA), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) and poly(ε-caprolactone) or mixtures of two or more of the foregoing. The biodegradable polymeric materials may be substantially pure single polymer or mixes of two or more polymers wherein the materials comprise mixtures of single monomers, block co-polymers or a mixture thereof.

Therapeutic compound(s)/agent(s) or mixtures of two or more therapeutic compound(s)/agent(s) can be formulated in a carrier system. The carrier can be a colloidal system. The carrier or colloidal system can be a liposome, a phospholipid bilayer vehicle. In one embodiment, therapeutic compound(s)/agent(s) or mixtures of two or more therapeutic compound(s)/agent(s) can be encapsulated in a liposome while maintaining integrity of the therapeutic compound(s)/agent(s). One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). For example, a therapeutic agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic compound(s)/agent(s) or mixtures of two or more therapeutic compound(s)/agent(s) can be embedded in the polymer matrix, while maintaining integrity of the composition. The polymer can be a microparticle or nanoparticle that encapsulates therapeutic compound(s)/agent(s). The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In some embodiments, the polymer is poly-lactic acid (PLA), poly lactic/glycolic acid (PLGA) or a mixture thereof. The polymeric matrices can be prepared and isolated in a variety of forms and sizes, including microspheres and nanospheres. Polymer formulations can lead to prolonged duration of therapeutic effect. (See Reddy, Ann. Pharmacother., 34 (7-8): 915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the nanoparticles or microparticles can be silica-based or silane-based (See for example: WO2002/080977 entitled: “Biodegradable carrier and method for preparation thereof”).

In some embodiments, the therapeutic compound(s)/agent(s) or mixtures of two or more therapeutic compound(s)/agent(s) can be prepared with carriers that will protect the therapeutic compound(s)/agent(s) or other therapeutic agent(s) or mixtures thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compound(s)/agent(s) or mixtures of two or more therapeutic compound(s)/agent(s) may be contained in controlled release systems. The term “controlled-release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained-release” (also referred to as “extended-release”) is used in its conventional sense to refer to a drug formulation that provides for gradual release of a drug over an extended period of time, and that preferably, although not necessarily, results in substantially constant blood levels of a drug over an extended time period. The term “delayed-release” is used in its conventional sense to refer to a drug formulation in which there is a time delay between administration of the formulation and the release of the drug therefrom to thereby make it available to the subject. “Delayed release” may or may not involve gradual release of drug over an extended period of time, and thus may or may not be “sustained release.”

Use of a long-term controlled-release or sustained-release implant or depot formulation may be particularly suitable for treatment of chronic conditions. The term “implant” and “depot formulation” is intended to include a single composition (such as a mesh) or composition comprising multiple components (e.g., a fibrous mesh constructed from several individual pieces of mesh material) or a plurality of individual compositions where the plurality remains localized and provides the long-term sustained-release of active pharmaceutical ingredient(s) occurring from the aggregate of the one or plurality of compositions. “Long-term” release, as used herein, means that the implant or depot formulation is constructed and arranged to deliver therapeutic or prophylactic levels of the active pharmaceutical ingredient(s) for at least 2 days. In some embodiments, the implant or depot formulation is constructed and arranged to deliver therapeutic or prophylactic levels of the active pharmaceutical ingredient(s) for at least 7 days. In some embodiments, the implant or depot formulation is constructed and arranged to deliver therapeutic or prophylactic levels of the active pharmaceutical ingredient(s) for at least 14 days. In some embodiments, the implant or depot formulation is constructed and arranged to deliver therapeutic or prophylactic levels of the active pharmaceutical ingredient(s) for at least 30 days. In some embodiments, the implant or depot formulation is constructed and arranged to deliver therapeutic or prophylactic levels of the active pharmaceutical ingredient(s) for at least 60 days. In some embodiments, the implant or depot formulation is constructed and arranged to deliver therapeutic or prophylactic levels of the active pharmaceutical ingredient for at least 90 days. In some embodiments, the implant or depot formulation is constructed and arranged to deliver therapeutic or prophylactic levels of the active pharmaceutical ingredient(s) for at least 180 days. In some embodiments, the implant or depot formulation is constructed and arranged to deliver therapeutic or prophylactic levels of the active pharmaceutical ingredient(s) for at least one year. In some embodiments, the implant or depot formulation is constructed and arranged to deliver therapeutic or prophylactic levels of the active pharmaceutical ingredient(s) for 15-30 days. In some embodiments, the implant or depot formulation is constructed and arranged to deliver therapeutic or prophylactic levels of the active pharmaceutical ingredient(s) for 30-60 days. In some embodiments, the implant or depot formulation is constructed and arranged to deliver therapeutic or prophylactic levels of the active pharmaceutical ingredient(s) for 60-90 days. In some embodiments, the implant or depot formulation is constructed and arranged to deliver therapeutic or prophylactic levels of the active pharmaceutical ingredient(s) for 90-120 days. In some embodiments, the implant or depot formulation is constructed and arranged to deliver therapeutic or prophylactic levels of the active pharmaceutical ingredient(s) for 120-180 days. In some embodiments, the implant or depot formulation is constructed and arranged to deliver therapeutic or prophylactic levels of the active pharmaceutical ingredient(s) for up to one year. In some embodiments, the long-term sustained-release implants or depot formulation are well-known to those of ordinary skill in the art and include some of the release systems described above. In some embodiments, such implants or depot formulation can be administered surgically. In some embodiments, such implants or depot formulation can be administered topically or by injection.

### III. Formulations and Medicaments

Compounds 1 to 4, or a mixture of any two or more of Compounds 1 to 4 can be used, alone or in combination, with one or more other therapeutic agents to address the needs of subjects suffering from Leigh syndrome. In order to be administered to a subject in need thereof, Compounds 1 to 4, or a mixture of any two or more of Compounds 1 to 4 and/or other therapeutic agent(s) will generally need to be formulated for the suitable route of administration. For example, if Compounds 1 to 4, or a mixture of any two or more of Compounds 1 to 4 and/or other therapeutic agent(s) is/are to be administered to the subject by injection, it/they will typically be formulated into an injectable liquid or liquid suspension. For example, this could be accomplished by dissolving or suspending the therapeutic agent(s) in a suitable diluent, adjuvant, excipient, vehicle or pharmaceutically acceptable carrier as described previously herein (See the section above entitled: Pharmaceutical Compositions, Routes of Administration, and Dosing). In some embodiments, the diluent, adjuvant, excipient, vehicle or pharmaceutically acceptable carrier can be water, saline or a buffered aqueous solution. Suitable methods, reagents and compositions for formulating Compounds 1 to 4, or a mixture of any two or more of Compounds 1 to 4 and/or other therapeutic agent(s) into a suitable medicament are discussed above.

Similarly, if Compounds 1 to 4, or a mixture of any two or more of Compounds 1 to 4 and/or other therapeutic agent(s) can be to be administered to the subject in oral form, the selected active ingredient(s) can be formulated into a pill, tablet, capsule or other vehicle for such administration as discussed above in the section entitled: “Pharmaceutical Compositions, Routes of Administration, and Dosing” or as otherwise known to those of ordinary skill in the art. Suitable methods, reagents and compositions for formulating Compounds 1 to 4, or a mixture of any two or more of Compounds 1 to 4 and/or other therapeutic agent(s) into a suitable orally administrable medicament are discussed above.

Similarly, Compounds 1 to 4, or a mixture of any two or more of Compounds 1 to 4 and/or other therapeutic agent(s) can be formulated for ocular administration, buccal administration, topical administration, nasal administration or any other of the modes of administration previously discussed herein or that are known to those of ordinary skill in the art. Suitable methods, reagents and compositions for formulating Compounds 1 to 4, or a mixture of any two or more of Compounds 1 to 4 and/or other therapeutic agent(s) into a suitable ocular, buccal, topical, or nasal administrable medicament are discussed above.

In brief, any of the formulations (which can also be referred to as a medicament or composition when formulated for administration to a subject having a certain affliction or medical condition that requires medical attention) described in the section above entitled: “Pharmaceutical Compositions, Routes of Administration, and Dosing” can be applied to produce a composition (i.e. a formulation or medicament) suitable for administration to a subject in need thereof. Thus, in some embodiments, this application is directed to compositions, formulations and medicaments suitable for administration to a subject suffering from, or believed to be suffering from, Leigh syndrome.

In some embodiments, the composition, formulation, or medicament is administered subcutaneously. In some embodiments, the composition, formulation, or medicament is administered orally, topically, intranasally, systemically, intravenously, intraperitoneally, intradermally, intraocularly, ophthalmically, intrathecally, intracerebroventricularly, iontophoretically, transmucosally, intravitreally, or intramuscularly.

In some embodiments, Compounds 1 to 4, and/or other therapeutic agent(s) can be administered in formulations prepared using a pharmaceutically acceptable salt form. In some embodiments, Compounds 1 to 4, and/or other therapeutic agent(s) can be administered in formulations prepared using a hydrate form. In some embodiments, Compounds 1 to 4, and/or other therapeutic agent(s) can be administered in formulations prepared using a solvated form. In some embodiments, Compounds 1 to 4, and/or other therapeutic agent(s) can be administered in formulations prepared using a tautomeric form. In some embodiments, Compounds 1 to 4, and/or other therapeutic agent(s) can be administered in formulations prepared using a stereoisomer of Compounds 1 to 4, and/or the other therapeutic agent(s).

### IV. Therapeutic Methods and Related Uses of the Disclosed Compounds in Addressing Leigh Syndrome

In one aspect, the present disclosure provides a treating, preventing, ameliorating, inhibiting or delaying the onset of Leigh syndrome, or its associated signs or symptoms, in a subject in need thereof, comprising administering to said subject a therapeutically effective amount of Compound 1, Compound 2, Compound 3, Compound 4, or a mixture of any two or more of Compounds 1 to 4, or pharmaceutically acceptable salts, stereoisomers, mixtures of stereoisomers, tautomers, hydrates, and/or solvates thereof, wherein Compounds 1 to 4 have the following structures:

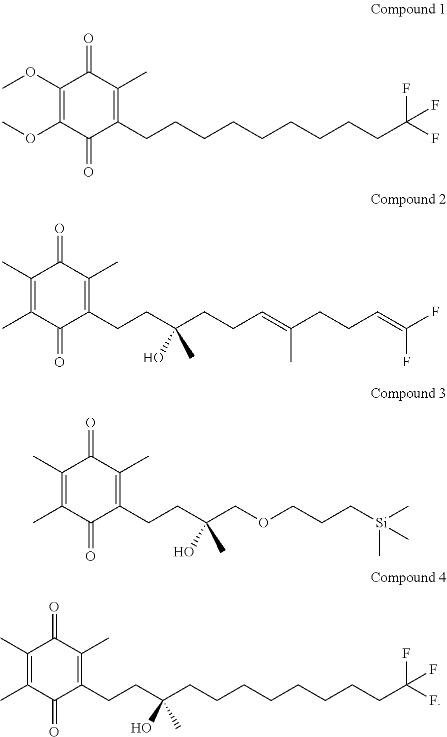

In some embodiments of the method, the signs or symptoms of Leigh syndrome include difficulty swallowing, difficulty breathing, difficulty eating, hypotonia, lactic acidosis, developmental delays, cardiomyopathy, seizures, ataxia, dysarthria, dystonia, paralysis, impairment of respiratory and kidney function, color blindness and/or vision loss. In some embodiments of the method, the subject has been diagnosed with Leigh syndrome. In some embodiments of the method, the subject is human.

In some embodiments of the method, the Compound(s) (i.e., Compound 1, Compound 2, Compound 3, Compound 4, or a mixture of any two or more of Compounds 1 to 4, or pharmaceutically acceptable salts, stereoisomers, mixtures of stereoisomers, tautomers, hydrates, and/or solvates thereof) is/are administered orally, topically, intranasally, systemically, intravenously, subcutaneously, intraperitoneally, intradermally, intraocularly, ophthalmically, intrathecally, intracerebroventricularly, iontophoretically, transmucosally, intravitreally, or intramuscularly. In some embodiments of the method, the Compound(s) is/are administered daily for 6 weeks or more.

In some embodiments of the method, administration of the Compound(s) (i.e., Compound 1, Compound 2, Compound 3, Compound 4, or a mixture of any two or more of Compounds 1 to 4, or pharmaceutically acceptable salts, stereoisomers, mixtures of stereoisomers, tautomers, hydrates, and/or solvates thereof) to the subject treats, prevents, ameliorates, inhibits or delays the onset of a cardiomyopathy in the subject. In some embodiments of the method, the cardiomyopathy is hypertrophic cardiomyopathy, left ventricular hypertrophy, dilated cardiomyopathy, pericardial effusions and/or an arrhythmia/conduction abnormality. In some embodiments of the method, the cardiomyopathy is hypertrophic cardiomyopathy.

In some embodiments of the method, administration of the Compound(s) (i.e., Compound 1, Compound 2, Compound 3, Compound 4, or a mixture of any two or more of Compounds 1 to 4, or pharmaceutically acceptable salts, stereoisomers, mixtures of stereoisomers, tautomers, hydrates, and/or solvates thereof) to the subject lowers lactic acid levels in the subjects blood, urine or cerebrospinal fluid (CSF).

In some embodiments of the method, administration of the Compound(s) (i.e., Compound 1, Compound 2, Compound 3, Compound 4, or a mixture of any two or more of Compounds 1 to 4, or pharmaceutically acceptable salts, stereoisomers, mixtures of stereoisomers, tautomers, hydrates, and/or solvates thereof) to the subject treats, prevents, ameliorates, inhibits or delays the onset of multi-organ and/or central nervous system (CNS) damage, including lesions in the brainstem, basal ganglia, and spinal cord of the subject.

In some embodiments of the method, administration of the Compound(s) (i.e., Compound 1, Compound 2, Compound 3, Compound 4, or a mixture of any two or more of Compounds 1 to 4, or pharmaceutically acceptable salts, stereoisomers, mixtures of stereoisomers, tautomers, hydrates, and/or solvates thereof) to the subject treats, prevents, ameliorates, inhibits or delays the onset of color blindness and/or vision loss of the subject.

In some embodiments of the method, administration of the Compound(s) (i.e., Compound 1, Compound 2, Compound 3, Compound 4, or a mixture of any two or more of Compounds 1 to 4, or pharmaceutically acceptable salts, stereoisomers, mixtures of stereoisomers, tautomers, hydrates, and/or solvates thereof) to the subject increases the lifespan of the subject.

In some embodiments of the method, administration of the Compound(s) (i.e., Compound 1, Compound 2, Compound 3, Compound 4, or a mixture of any two or more of Compounds 1 to 4, or pharmaceutically acceptable salts, stereoisomers, mixtures of stereoisomers, tautomers, hydrates, and/or solvates thereof) to the subject treats, prevents, ameliorates, inhibits or delays the onset of seizures, ataxia, dysarthria, dystonia and/or paralysis of the subject.

In another aspect, present disclosure provides a composition, medicament or formulation comprising any one of Compounds 1 to 4, or a mixture of any two or more of Compounds 1 to 4, or pharmaceutically acceptable salts, stereoisomers, mixtures of stereoisomers, tautomers, hydrates, and/or solvates thereof for use in treating, preventing, ameliorating, inhibiting, or delaying the onset of Leigh syndrome, or its associated signs or symptoms in a subject in need thereof, wherein Compounds 1 to 4 have the following structures:

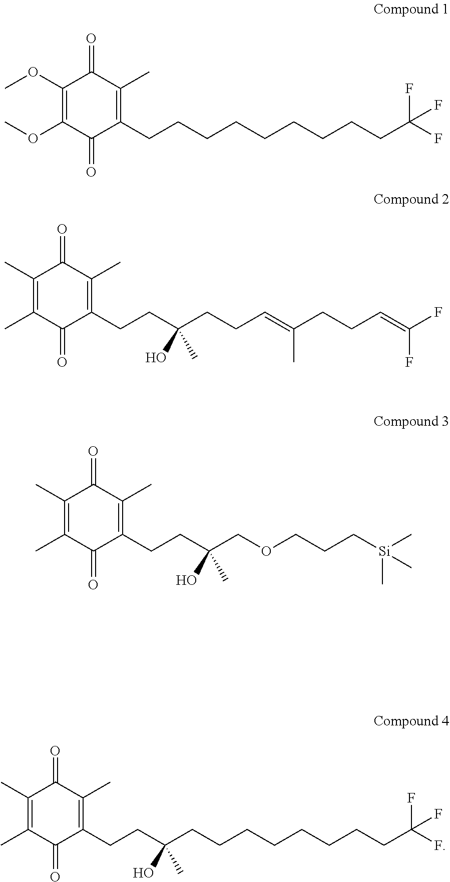

In some embodiments of the composition, medicament or formulation, the signs or symptoms of Leigh syndrome include difficulty swallowing, difficulty breathing, difficulty eating, hypotonia, lactic acidosis, developmental delays, cardiomyopathy, seizures, ataxia, dysarthria, dystonia, paralysis, impairment of respiratory and kidney function, color blindness and/or vision loss. In some embodiments of the composition, medicament or formulation, the subject has been diagnosed with Leigh syndrome. In some embodiments of the composition, medicament or formulation, the subject is human.

In some embodiments, the composition, medicament or formulation comprising the Compound(s) (i.e., Compound 1, Compound 2, Compound 3, Compound 4, or a mixture of any two or more of Compounds 1 to 4, or pharmaceutically acceptable salts, stereoisomers, mixtures of stereoisomers, tautomers, hydrates, and/or solvates thereof) is/are administered orally, topically, intranasally, systemically, intravenously, subcutaneously, intraperitoneally, intradermally, intraocularly, ophthalmically, intrathecally, intracerebroventricularly, iontophoretically, transmucosally, intravitreally, or intramuscularly. In some embodiments, the composition, medicament or formulation comprising the Compound(s) is/are administered daily for 6 weeks or more.

In some embodiments, administration of the composition, medicament or formulation comprising the Compound(s) (i.e., Compound 1, Compound 2, Compound 3, Compound 4, or a mixture of any two or more of Compounds 1 to 4, or pharmaceutically acceptable salts, stereoisomers, mixtures of stereoisomers, tautomers, hydrates, and/or solvates thereof) to the subject treats, prevents, ameliorates, inhibits or delays the onset of a cardiomyopathy in the subject. In some embodiments, the cardiomyopathy is hypertrophic cardiomyopathy, left ventricular hypertrophy, dilated cardiomyopathy, pericardial effusions and/or an arrythmia/conduction abnormality. In some embodiments, the cardiomyopathy is hypertrophic cardiomyopathy.

In some embodiments, administration of the composition, medicament or formulation comprising the Compound(s) (i.e., Compound 1, Compound 2, Compound 3, Compound 4, or a mixture of any two or more of Compounds 1 to 4, or pharmaceutically acceptable salts, stereoisomers, mixtures of stereoisomers, tautomers, hydrates, and/or solvates thereof) to the subject lowers lactic acid levels in the subjects blood, urine or cerebrospinal fluid (CSF).

In some embodiments, administration of the composition, medicament or formulation comprising the Compound(s) (i.e., Compound 1, Compound 2, Compound 3, Compound 4, or a mixture of any two or more of Compounds 1 to 4, or pharmaceutically acceptable salts, stereoisomers, mixtures of stereoisomers, tautomers, hydrates, and/or solvates thereof) to the subject treats, prevents, ameliorates, inhibits or delays the onset of multi-organ and/or central nervous system (CNS) damage, including lesions in the brainstem, basal ganglia, and spinal cord of the subject.

In some embodiments, administration of the composition, medicament or formulation comprising the Compound(s) (i.e., Compound 1, Compound 2, Compound 3, Compound 4, or a mixture of any two or more of Compounds 1 to 4, or pharmaceutically acceptable salts, stereoisomers, mixtures of stereoisomers, tautomers, hydrates, and/or solvates thereof) to the subject treats, prevents, ameliorates, inhibits or delays the onset of color blindness and/or vision loss of the subject.

In some embodiments, administration of the composition, medicament or formulation comprising the Compound(s) (i.e., Compound 1, Compound 2, Compound 3, Compound 4, or a mixture of any two or more of Compounds 1 to 4, or pharmaceutically acceptable salts, stereoisomers, mixtures of stereoisomers, tautomers, hydrates, and/or solvates thereof) to the subject increases the lifespan of the subject.

In some embodiments, administration of the composition, medicament or formulation comprising the Compound(s) (i.e., Compound 1, Compound 2, Compound 3, Compound 4, or a mixture of any two or more of Compounds 1 to 4, or pharmaceutically acceptable salts, stereoisomers, mixtures of stereoisomers, tautomers, hydrates, and/or solvates thereof) to the subject treats, prevents, ameliorates, inhibits or delays the onset of seizures, ataxia, dysarthria, dystonia and/or paralysis of the subject.

In another aspect, present disclosure provides use for Compounds 1 to 4, or a mixture of any two or more of Compounds 1 to 4, or pharmaceutically acceptable salts, stereoisomers, mixtures of stereoisomers, tautomers, hydrates, and/or solvates thereof in preparation of a composition, formulation or medicament comprising any one of Compounds 1 to 4, or a mixture of any two or more of Compounds 1 to 4, or pharmaceutically acceptable salts, stereoisomers, mixtures of stereoisomers, tautomers, hydrates, and/or solvates thereof in treating, preventing, ameliorating, inhibiting, or delaying the onset of Leigh syndrome, or its associated signs or symptoms, in a subject in need thereof, wherein Compounds 1 to 4 have the following structures:

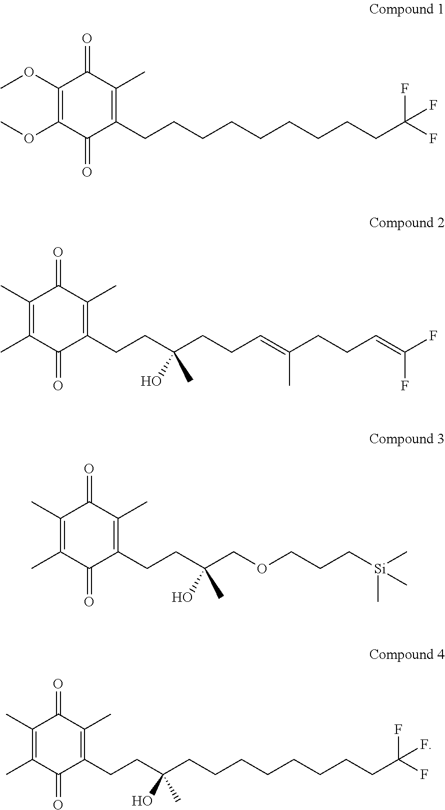

In some embodiments of the uses, the signs or symptoms of Leigh syndrome include difficulty swallowing, difficulty breathing, difficulty eating, hypotonia, lactic acidosis, developmental delays, cardiomyopathy, seizures, ataxia, dysarthria, dystonia, paralysis, impairment of respiratory and kidney function, color blindness and/or vision loss. In some embodiments of the uses, the subject has been diagnosed with Leigh syndrome. In some embodiments of the uses, the subject is human.

In some embodiments of the uses, the Compound(s) (i.e., Compound 1, Compound 2, Compound 3, Compound 4, or a mixture of any two or more of Compounds 1 to 4, or pharmaceutically acceptable salts, stereoisomers, mixtures of stereoisomers, tautomers, hydrates, and/or solvates thereof, including compositions, formulations or medicaments derived therefrom) is/are administered orally, topically, intranasally, systemically, intravenously, subcutaneously, intraperitoneally, intradermally, intraocularly, ophthalmically, intrathecally, intracerebroventricularly, iontophoretically, transmucosally, intravitreally, or intramuscularly. In some embodiments of the uses, the Compound(s) is/are administered daily for 6 weeks or more.

In some embodiments of the uses, administration of the compositions, formulations or medicaments to the subject treats, prevents, ameliorates, inhibits or delays the onset of a cardiomyopathy in the subject. In some embodiments of the uses, the cardiomyopathy is hypertrophic cardiomyopathy, left ventricular hypertrophy, dilated cardiomyopathy, pericardial effusions and/or an arrythmia/conduction abnormality. In some embodiments of the uses, the cardiomyopathy is hypertrophic cardiomyopathy.

In some embodiments of the uses, administration of the compositions, formulations or medicaments to the subject lowers lactic acid levels in the subjects blood, urine or cerebrospinal fluid (CSF).

In some embodiments of the uses, administration of the compositions, formulations or medicaments to the subject treats, prevents, ameliorates, inhibits or delays the onset of multi-organ and/or central nervous system (CNS) damage, including lesions in the brainstem, basal ganglia, and spinal cord of the subject.

In some embodiments of the uses, administration of the compositions, formulations or medicaments to the subject treats, prevents, ameliorates, inhibits or delays the onset of color blindness and/or vision loss of the subject.

In some embodiments of the uses, administration of the compositions, formulations or medicaments to the subject increases the lifespan of the subject.

In some embodiments, administration of the composition, medicament or formulation to the subject treats, prevents, ameliorates, inhibits or delays the onset of seizures, ataxia, dysarthria, dystonia and/or paralysis of the subject.

### EXAMPLES

The present technology is further illustrated by the following examples, which should not be construed as limiting in any way.

### Example 1: In Vitro Assays

### I. Materials & Methods

### Cell Lines:

Patient fibroblasts were acquired from Coriell Institute for Medical Research (Camden, NJ, USA). GM03672 is an untransformed fibroblast cell line from a Leigh syndrome patient carrying an unknown mutation. GM08402 is an untransformed fibroblast from an apparently healthy individual. Cells were cultured at 37° C. at 5% CO₂ in Eagle's Minimum Essential Medium (MEM, Corning), which was supplemented with Earle's salts and non-essential amino acids, 15% qualified fetal bovine serum, and 1% penicillin/streptomycin (100 U/100 g) to yield “Complete MEM.”

### Mitochondria Isolation:

For mitochondria isolation, brains were dissected at a local vivarium (Explora, MA, USA) from C57BL/6 mice that were supplied by The Jackson Laboratory (Maine, USA). Brains were collected on ice in cold mitochondria isolation buffer (225 mM mannitol, 75 mM sucrose, 5 mM HEPES, 1 mM EGTA, pH 7.4). In a glass petri dish, brains were mixed with a few drops of buffer and cut into small pieces resembling a mash using scissors. Mitochondrial isolation buffer was added at 1:10-1:20 (w/v) and tissue mash was transferred to a glass Dounce tissue homogenizer and further homogenized with 10-15 strokes. Mash was centrifuged at 1,000×g for 10 min to pellet and remove cellular debris, etc. Supernatant was transferred into 2.0 mL tubes and centrifuged at 10,000×g. The mitochondria pellet was resuspended in the desired amount of miR05 buffer (Oroboros, Innsbruck, Austria) and added directly to the Oroboros oxygraph-2K (Oroboros, Innsbruck, Austria) for an experiment or maintained on ice for up to 4 hours.

### High-Resolution Respirometry (HRR):

All high-resolution respirometry experiments were conducted using the Oroboros O2k-Fluorespirometer (Oroboros, Innsbruck, Austria). The Oroboros O2k-Fluorespirometer was calibrated daily using the air calibration protocol on DatLab 7.4 (Oroboros, Innsbruck, Austria). Prior to each experiment requiring the fluorescence module, the AmpR calibration protocol from DatLab 7.4 was used to calibrate the fluorescence signal. Initial data analysis, including setting of marks, was performed using DatLab 7.4.

### Complex I-Bypass Assay ((CI)-Bypass Assay):

Complex I-bypass activity was assessed using HRR to measure O₂ consumption. Briefly, Leigh (GM03672) fibroblasts were harvested with trypsin, pelleted at 300×g centrifugation, and resuspended in prewarmed, complete MEM. Cells were added to each chamber via buffer replacement at a concentration of 1-2 million per mL. The chambers were closed to initiate measurement of O₂ consumption. The slope negative signal, which is a derivative of O₂ consumption, was allowed to stabilize over the course of several minutes. Using a Hamilton syringe to allow for continuous measurement of O₂ consumption, glutamate (10 mM final concentration) and malate (0.1 mM final concentration) were added to the closed chamber through the central capillary of the stopper. The slope negative signal was stabilized for several minutes, and rotenone was added at a final concentration of 0.5 μM. One of Compounds 1 to 4 or Vatiquinone was added at increasing concentrations, allowing for several minutes of stabilization of the slope negative signal between titrations. Marks were set at signal plateaus in between titrations. Values were exported and analyzed using GraphPad Prism 9. Vatiquinone data is not shown, as it displayed no CI-bypass activity at concentrations in range of those used for Compounds 1 to 4 (i.e., titrations between 125 nM and 8 μM). Responsiveness of cells was confirmed with idebenone.

Structure of Vatiquinone for reference purposes:

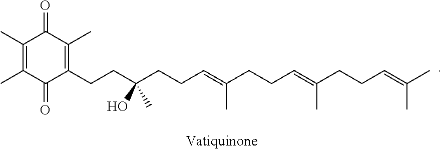 [SMILES: CC(C)=CCC/C(C)=C/CC/C(C)=C/CC[C@@](C)(O)CCC1=C(C)C(=O)C(C)=C(C)C1=O]

### Reverse Electron Transfer Assay (RET Assay):

Reverse electron transfer (RET) was measured using HRR with the Oroboros O2k and DatLab 7.4. To configure the Amp channel, the light or “Fluo intensity” was set at 1000 and the amplification of signal or “Gain for Fluo sensor” at 500 and illumination in the chamber was turned “off”. To calibrate the Fluo sensors, the Instrument protocol “AmR calibration” was used. Briefly, chambers were loaded with miR05 buffer and the following reagents were added at the final concentrations listed: 15 μM DTPA, 5 U/mL superoxide dismutase (SOD), 1 U/mL horseradish peroxidase (HRP) and 10 μM Amplex Ultra Red. A three-point calibration curve was derived from two, 5 L-injections of 40 μM H₂O₂ and used to calibrate each chamber prior to beginning the experiment. Mitochondria were suspended in pre-warmed, complete miR05 buffer at a concentration of approximately 1,500 μg per mL. To each chamber, 100 μL of mitochondria suspension was added through the central capillary of the chamber stopper using a Hamilton syringe. Routine respiration was established for several minutes. Sufficient amounts of one of Compounds 1 to 4 from stock vials were added to yield final concentration of 1 or 2 μM as indicated. “No treatment” controls did not receive any of Compounds 1 to 4. Prior studies established no effect of the drug vehicle on mitochondria respiration. The slope negative signal was allowed to stabilize for several minutes before the addition of succinate (10 mM final concentration), a Complex II substrate, which generates reactive oxygen species (ROS) through the reverse transfer of electrons to Complex I. Following the addition of succinate (10 mM final concentration), the slope negative signal was allowed to stabilize for several minutes. Marks were made on the trace of the slope negative of H₂O₂ as appropriate to determine H₂O₂ production (a form of ROS). Values were exported to GraphPad Prism 9 (GraphPad Software, Boston, MA) for analysis.

### RSL3 Cytoprotection Assay (Cytoprotection Assay):

To initiate the RSL3 cytoprotection experiment, Healthy (GM08402) and Leigh syndrome (GM03672) fibroblasts were resuspended in Complete MEM. Cells were seeded at 10,000 cells per well in a clear, tissue cultured-treated, 96-well plate and incubated at 37° C. overnight. Following the incubation, supernatant was manually aspirated, taking care not to disrupt cells. Healthy fibroblasts were co-treated in a final volume of 100 μL with 1000 nM (1S,3R)-Methyl 2-(2-chloroacetyl)-2,3,4,9-tetrahydro-1-[4-(methoxycarbonyl)phenyl]-1H-pyrido[3,4-b]indole-3-carboxylate (RSL3) and a titration of Compounds 1 to 4 or Vatiquinone (Cayman Chemical, Ann Arbor, Michigan) ranging from 10 and 1000 nM. Leigh fibroblasts were co-treated in a final volume of 100 μL with either 375 nM or 700 nM RSL3 and a titration of one of Compounds 1 to 4 or Vatiquinone ranging from 10 and 1000 nM. Cells were incubated for 4 hours at 37° C. Cell viability was measured using the CellTiter-Glo Luminescent Cell Viability Kit (ProMega) per Manufacturer's instructions. Luminescence units were quantified by GEN5 software on a BioTek SYNERGY neo2 plate reader (LUM filter).

### 15(S)-HETE ELISA Following RSL3 Treatment (HETE ELISA/RSL3 Assay):

Samples for the 15(S)-HETE ELISA were generated by the RSL3 cytoprotection assay described above. Healthy or Leigh fibroblasts were resuspended in assay medium, seeded at 200,000 cells per well in a clear, tissue culture-treated, 24-well plate. Cells were incubated at 37° C. for 24 hours. Supernatant was aspirated from each well following the incubation. Fibroblasts were co-treated with 700 nM RSL3, and 1 μM of one or Compounds 1 to 4 or Vatiquinone in a final volume of 0.4 mL and incubated for 24 hours at 37° C. The concentration of 15(S)-HETE was measured in the supernatant using Abcam's 15(S) HETE-ELISA Kit (Cat #ab 133035) per Manufacturer's instructions. Absorbance at 405 nM was measured by GEN5 software on a BioTek SYNERGY neo2 plate reader.

### Erastin Cytoprotection Assay (Erastin Assay)

Briefly, Healthy (GM08402) or Leigh (GM03672) fibroblasts were resuspended in assay medium, seeded at 10,000 cells per well in a clear, tissue cultured-treated, 96-well plate, and incubated at 37° C. overnight. Following the incubation, supernatant was manually aspirated from wells, taking care not to disrupt cells. Fibroblasts were co-treated in a final volume of 100 μL with 2 μM erastin (Millipore Corp., Cat #), 250 μM ferric ammonium citrate (FAC), 50 μM ascorbic acid (AA), and a titration of one of each of Compounds 1 to 4 or Vatiquinone ranging from 10 to 1000 nM. Following another 24-hour incubation at 37° C., cell viability was measured using the CellTiter-Glo Luminescent Cell Viability Kit (ProMega) per Manufacturer's instructions. Luminescence units were quantified by GEN5 software on a BioTek SYNERGY neo2 plate reader (LUM filter).

### 15(S)-HETE ELISA Following Erastin Treatment (HETE ELISA/Erastin Assay)

Samples for the 15(S)-HETE ELISA were generated by the Erastin Assay described above. Healthy or Leigh fibroblasts were resuspended in assay medium, seeded at 200,000 cells/well in a clear, tissue culture-treated, 24-well plate, and incubated at 37° C. for 24 hours. Supernatant was aspirated from each well. Fibroblasts were co-treated with 1 μM erastin (Millipore Corp, Cat #), 250 μM ferric ammonium citrate (FAC), 50 μM ascorbic acid (AA), and 1 μM of one of Compounds 1 to 4 in a final volume of 0.4 mL and incubated for 24 hours at 37° C. The concentration of 15(S)-HETE in the supernatant were measured using Abcam's 15(S)-HETE-ELISA Kit (Cat #ab 133035). Absorbance at 405 nM was measured by GEN5 software on a BioTek SYNERGY neo2 plate reader.

### Statistical Analysis

Statistical analysis was performed using GraphPad Prism 9. Complex I-bypass data were fit using nonlinear sigmoidal 4P fit. RET Assay data were analyzed by 2-way ANOVA using a p-value of 0.05.

### II. Results

### A. ((CI)-Bypass Assay)

The Complex 1-bypass assay ((CI)-bypass Assay) evaluates the capacity of compounds to donate electrons into the electron transport chain (ETC) when Complex I is impaired or otherwise deficient. Normally, electrons feed into the ETC through Complex I. In instances where Complex I is impaired, electrons must enter the ETC downstream of Complex I for oxidative phosphorylation (OXPHOS) to occur. Complex I is composed of 46 subunits, encoded by mitochondrial and nuclear genes. Due to its large size, Complex I mutations are a common cause of ETC dysfunction in mitochondrial diseases. Indeed, mutations in the genes that encode subunits for Complex I are important causes of Leigh and Leigh-like syndrome.

Using this assay, the ability of Compounds 1 to 4 and Vatiquinone to bypass rotenone-inhibited Complex I was determined. Rotenone is a widely used Complex I inhibitor and blocks the transfer of electrons from the iron-sulfur complexes on Complex I to ubiquinone. Thus, when rotenone is introduced to cells underdoing routine respiration, a decrease in oxygen consumption is observed, which is indicative of disrupted OXPHOS. The ability of Compounds 1 to 4 and Vatiquinone to “bypass” Complex I and restore OXPHOS is measured by an increase in oxygen consumption rate following addition of the compound to the chamber.

In executing this assay, in each instance, fibroblasts are added to each chamber of the Oroboros Oxygraph-2K (the “O2k”). The O2k generates two traces: 1) total oxygen consumption over time, 2) the rate of oxygen consumption over time (slope negative). Baseline respiration is established for several minutes by allowing the slope negative to stabilize. Rotenone is introduced to each chamber at a final concentration of 0.5 μM. The addition of rotenone is expected to cause a marked reduction of OXPHOS as measured by a decreased rate of oxygen consumption. Then the test article (i.e., in this case one of Compounds 1 to 4 or Vatiquinone) is added to the chamber. If oxygen consumption resumes after addition on the test article, then the test article is presumed to allow for OXPHOS to continue though by-pass of the still rotenone-inhibited Complex I.

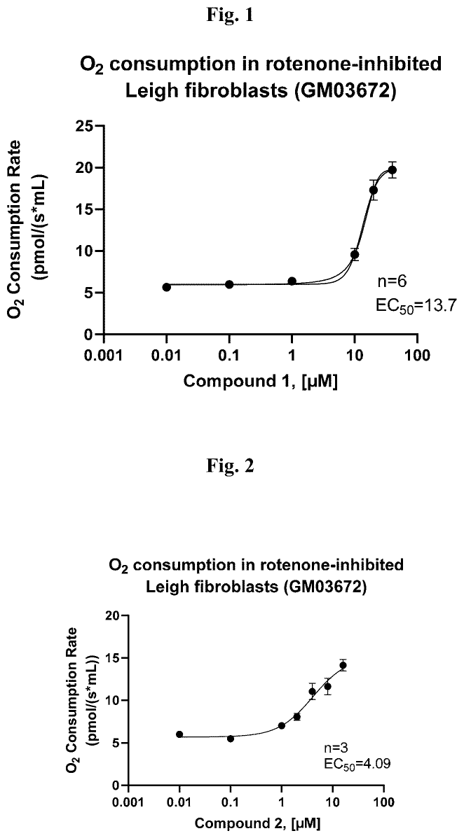

With reference to FIG. 1, the ability of Compound 1 to “bypass” the rotenone-inhibited Complex I is illustrated. The graph represents the aggregate of six experiments (n=6) in which Compound 1 was titrated into a chamber of rotenone-inhibited Leigh fibroblasts. The points on the graph correspond to “marks” that were made on the slope negative trace that was generated by the O2k. Marks were set on stable, or “flat” regions of the trace following addition of a given treatment, such as rotenone or a defined amount of test compound. Compound 1 restored oxygen consumption rate in a dose-dependent manner. The data for the six runs (i.e., n=6) were fit using a four-parameter sigmoidal curve using GraphPad Prism 9. The EC50 value for Compound 1 in the assay was determined to be approximately 13.7 μM.

Similarly, with Reference to FIG. 2, the ability of Compound 2 to “bypass” the rotenone-inhibited Complex I is illustrated. The assay was performed and data collected as described above, except the number of individual runs was 3 (i.e., n=3). An EC50 value for Compound 2 in the assay was determined to be approximately 4.09 μM.

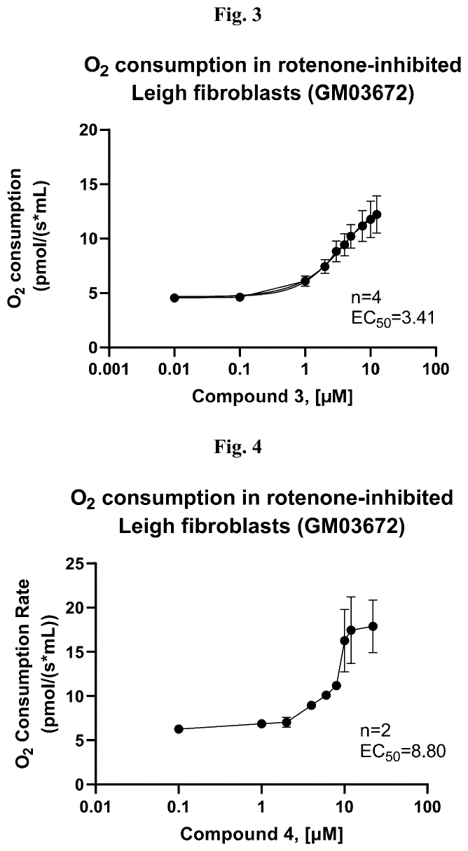

With Reference to FIG. 3, the ability of Compound 3 to “bypass” the rotenone-inhibited Complex I is illustrated. The assay was performed and data collected as described above, except the number of individual runs was 4 (i.e., n=4). An EC50 value for Compound 3 in the assay was determined to be approximately 3.41 μM.

With Reference to FIG. 4, the ability of Compound 4 to “bypass” the rotenone-inhibited Complex I is illustrated. The assay was performed and data collected as described above, except the number of individual runs was 2 (i.e., n=2). An EC50 value for Compound 4 in the assay was determined to be approximately 8.80 μM.

Vatiquinone was inactive in the Complex I by-pass assay at concentrations from 125 nM to 8 μM.

### B. RET Assay

The Reverse Electron Transfer Assay is a common assay in HRR that is used to generate reactive oxygen species (ROS). In this instance, the assay was performed using isolated murine brain mitochondria. In the absence of a Complex I inhibitor or substrate, introduction of succinate, which is a substrate for Complex II, results in reverse transfer of electrons onto Complex I. The reverse transfer of electrons generates ROS, which is measured through HRR using the O2k.

In the following experiments, the RET Assay was used to determine whether Compounds 1 to 4 could reduce the production of ROS. Reduction of ROS could occur through several mechanisms, which require further experimentation to elucidate. Reduction of H₂O₂ production, which is a proxy for ROS, indicates that the test article compound reduces ROS generated by reverse transfer of electrons. Reduction of ROS could indicate that the test article compound is acting as a scavenger or is enhancing electron flow through the electron transfer chain (ETC).

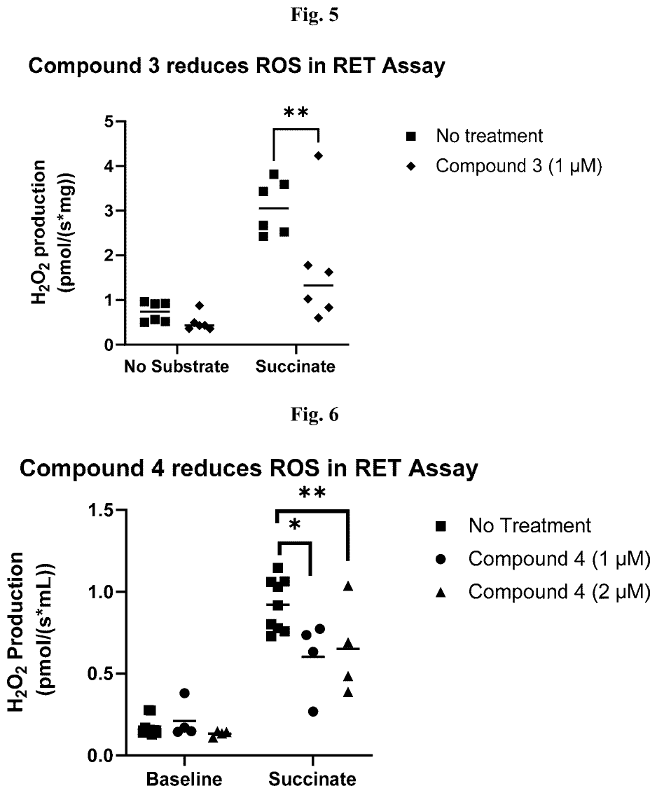

With Reference to FIG. 5, treatment of isolated mitochondria with 2 μM of Compound 3 prior to addition of succinate in the RET assay, resulted in statistically significant reduction in H₂O₂ production.

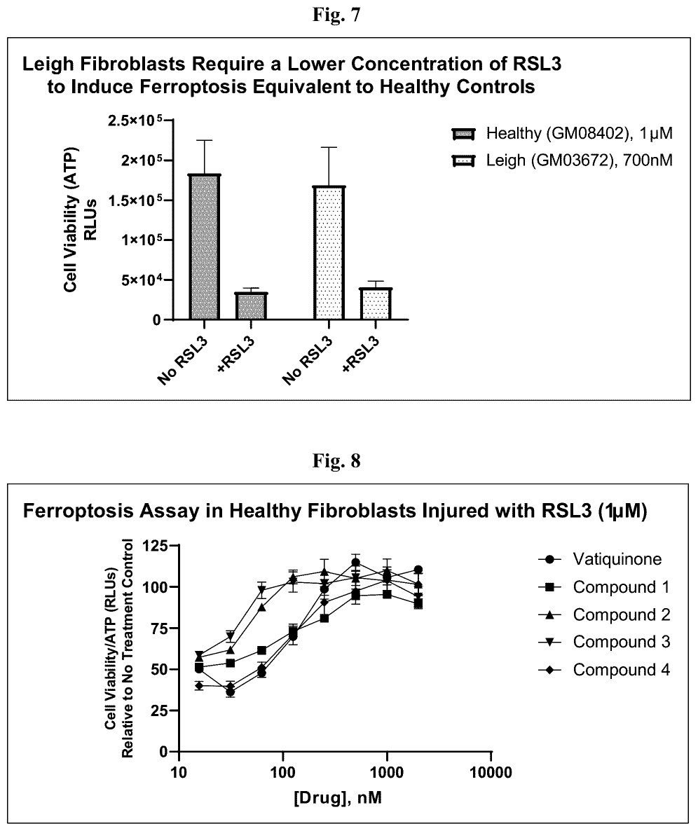

With Reference to FIG. 6, treatment of isolated mitochondria with either 1 or 2 μM of Compound 4 prior to the addition of succinate in the RET assay, resulted in statistically significant reduction in H₂O₂ production.

Compounds 1 and 2 did not produce results that could be interpreted in this assay. Vatiquinone was not tested in the RET assay.

### C. Cytoprotection Assay

RSL3 is an inhibitor of the enzyme glutathione peroxidase 4 (GPX4), which decreases lipid peroxides. RSL3 inhibition of GPX4 induces ferroptosis through iron-dependent accumulation of lipid ROS. In this assay, Healthy or Leigh fibroblasts were treated for 4 hours with RSL3 to induce ferroptosis or left untreated. Endogenous ATP levels were measured as an indicator of cellular viability using a commercially available kit (CellTiter-Glo® 2.0 Cell Viability Assay (Promega)).

Ferroptosis and 15-LO have been proposed in the field of mitochondrial research as rational targets for Leigh syndrome. Modeling of Leigh syndrome in mice suggests that impaired iron homeostasis contributes to disease pathogenesis, highlighting the importance of ferroptosis. Ferroptosis is also thought to be a driver of epilepsy. The 15-LO inhibitor Vatiquinone has been shown to reduce the incidence of seizures in patients with pontocerebellar hypoplasia type 6, a mitochondrial disorder that, like Leigh syndrome, is associated with epilepsy. Thus, it follows that disrupting ferroptosis could help decrease morbidity and disease pathogenesis in Leigh syndrome.

In this assay, the ability of Compounds 1 to 4 and Vatiquinone to inhibit/ameliorate/delay iron-mediated cell death from ferroptosis was analyzed. An increase in cell viability after addition of the test article compound indicates a protective effect against ferroptosis. Briefly, ferroptosis was induced with 1 μM RSL3 in healthy fibroblast cells and 375 nM RSL3 in fibroblasts from a patient with Leigh syndrome. Cells were cotreated with dimethyl sulfoxide (DMSO) or ethanol (EtOH; each a vehicle control), each of Compounds 1 to 4, or Vatiquinone. Each of Compounds 1 to 4 or Vatiquinone were administered at a range of concentrations to derive a titration curve. Cell viability was determined using a commercially-available kit that measures ATP. On the graph, the x-axis shows the range of drug concentrations used, while the y-axis shows Cell Viability (measured by ATP generation), as measured by relative luminescence units.

A preliminary experiment was performed to determine the effect of RSL3 injury on the fibroblasts of a healthy individual (cell line: GM08402) and the fibroblasts of a patient diagnosed with Leigh syndrome (cell line: GM03672). The data obtained demonstrated a differential sensitivity of the ferroptosis inducing agent (RSL3) on the fibroblasts of a healthy individual and the fibroblasts of a patient with Leigh syndrome. The data presented in FIG. 7 illustrates that roughly equivalent injury occurs when the fibroblasts from the healthy individual are treated with 1 μM RSL3 and the fibroblasts from the patient with Leigh syndrome are treated with 700 nM RSL3. These data suggest that to achieve an experimental window of 25-30% killing of cells, 1 μM RSL3 is sufficient in healthy fibroblasts while 700 nM is sufficient in Leigh fibroblasts.

With Reference to FIG. 8, this assay was performed on healthy cells. The test articles were Compounds 1 to 4, and Vatiquinone. Each of Compounds 1 to 4 and Vatiquinone produced a protective effect on the cells, thereby suggesting that each of Compounds 1 to 4 and Vatiquinone possess an anti-ferroptosis property. Additionally, the data suggest that Compounds 2 and 3 are roughly equivalent in their anti-ferroptosis properties and that they are superior to Compounds 1, 4 and Vatiquinone in their anti-ferroptosis properties (with Compounds 1, 4 and Vatiquinone being roughly equivalent in their anti-ferroptosis properties) with respect to treatment of these fibroblasts for a healthy individual in this particular assay.

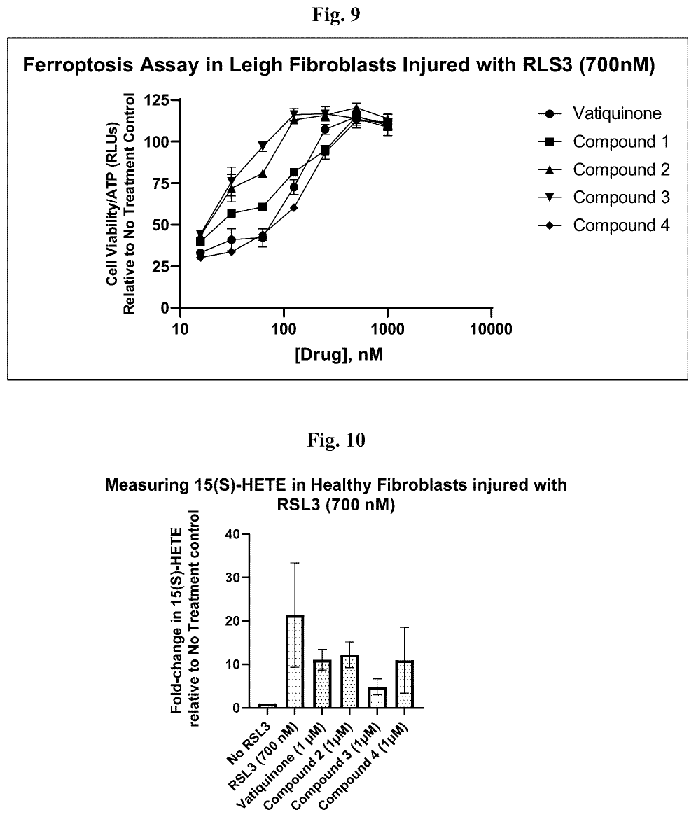

With Reference to FIG. 9, this assay was performed on fibroblasts from a patient with Leigh syndrome. The test articles were Compounds 1 to 4 and Vatiquinone. Each of Compounds 1 to 4 and Vatiquinone produced a protective effect on the cells, thereby suggesting that each of Compounds 1 to 4 and Vatiquinone possess an anti-ferroptosis property. Similarly (as observed in the fibroblasts from a healthy individual), the data suggest that Compounds 2 and 3 are roughly equivalent in their anti-ferroptosis properties and that they are superior to Compounds 1, 4 and Vatiquinone in their anti-ferroptosis properties (with Compounds 1, 4 and Vatiquinone being roughly equivalent in their anti-ferroptosis properties) with respect to treatment of these fibroblasts derived from a patient with Leigh syndrome in this assay. It is noteworthy that each of Compounds 1 to 4 and Vatiquinone were roughly similar in its relative effect in this assay regardless of whether the cell type was from a healthy individual or a patient diagnosed with Leigh syndrome.

In summary, regardless of the cell type examined, each of Compounds 1 to 4 and Vatiquinone produced similar results. Compounds 2 and 3 appear to be superior to Compounds 1, 4 and Vatiquinone in their ability to protect the (healthy or diseased) cells against RSL3 induced ferroptosis but all of Compounds 1 to 4 and Vatiquinone were found to be protective against RSL3 induced ferroptosis in cells from a healthy individual and from a patient diagnosed with Leigh syndrome.

### D. HETE ELISA/RSL3 Assay

This assay was used as an indirect way to determine the ability of each of Compounds 1 to 4 and Vatiquinone to inhibit 15-lipoxygenase (“15-LO”) using a commercially available ELISA assay format. 15(S)-HETE is a metabolite of arachidonic acid. Decreased 15(S)-HETE in the supernatant (as determined by the ELISA assay) suggests that 15-LO is being inhibited by the addition of a test article when compared with the no treatment control. Hence all data were normalized to the “No treatment” (i.e., no test article control) control group. Again, the ability of Compounds 1 to 4 and Vatiquinone were examined. In these assays, ferroptosis was induced by addition of RSL3 and the inhibition of 15-lipoxygenase (“15-LO”) attributable to each test article was determined was indirectly measured by measuring the output of 15(S)-HETE. Fibroblasts from both a healthy individual and a patient diagnosed with Leigh syndrome were examined (but not all compounds were examined in each cell type). Activation of ferroptosis has been implicated as a driver of epilepsy, which is commonly associated with Leigh syndrome. In pontocerebellar hypoplasia type 6, the 15-LO inhibitor Vatiquinone has been shown to decrease seizure frequency and morbidity. Kahn-Kirby et al. PLOS One. 2019; 14(3): e0214250. Published online 2019 Mar. 28. doi: 10.1371/journal.pone.0214250. As a key regulator of ferroptosis, 15-LO serves as a rational therapeutic target in Leigh syndrome.

With Reference to FIG. 10, the data derived using fibroblasts from a health individual that have been subjected to 700 nM RSL3 to induce ferroptosis are presented graphically. The data suggests that Compounds 2, 3 and 4 (Compound 1 not tested) and Vatiquinone had a protective effect in that they inhibited 15-lipoxygenase. In this experiment, Compound 3 appears to be superior to Compounds 2, 4 and Vatiquinone; each of which produces a roughly equivalent protective effect in this assay.

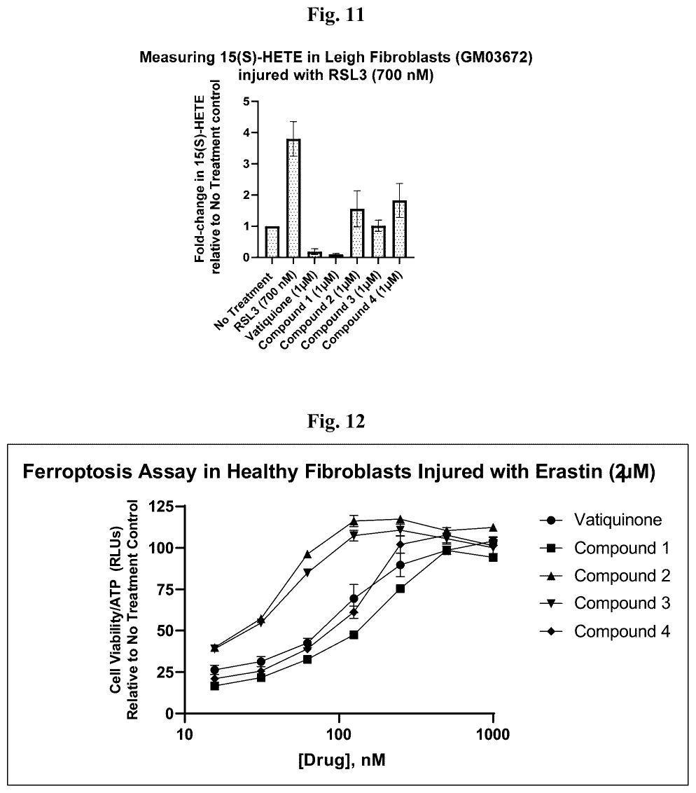

With Reference to FIG. 11, the data derived using fibroblasts from a patient diagnosed with Leigh syndrome that have been subjected to 700 nM RSL3 to induce ferroptosis are presented graphically. The data suggests that all of Compounds 1 to 4 and Vatiquinone had a protective effect (i.e., they inhibited 15-LO). In this assay, Vatiquinone and Compound 1 appear to be superior to Compounds 2, 3 and 4; each of which produces a roughly equivalent protective effect in this assay. It is noteworthy that the compounds tested appear to behave differently in this assay depending on cell type (i.e., healthy vs. diseased cells).

### E. Erastin Assay

Briefly, erastin, like RSL3, is known to induce ferroptosis. Hence, in this study, ferroptosis was induced by adding 2 μM erastin to the cell cultures. Erastin was incubated with cell fibroblasts for 24 hours to induce ferroptosis. Cells were co-treated with each of Compounds 1 to 4, or Vatiquinone as the test articles. Test articles were administered at a range of concentrations to derive a curve. Cell viability was determined using a commercially available kit that measures ATP production. In each graph provided, the x-axis shows the range of the test article concentrations used, while the y-axis shows Cell Viability (ATP production), as measured by relative luminescence units. Hence, this assay is used to determine if the test articles exhibit an anti-ferroptosis effect (i.e., a protective effect on the cells). An increase in cell viability as determined by the assay is considered to be protective against ferroptosis.

With Reference to FIG. 12, Compounds 1 to 4 and Vatiquinone were examined with respect to whether or not they were protective in fibroblasts from a healthy individual. In the assay, Compounds 2 and 3 appear to be superior in their protective effect as compared with Vatiquinone or Compounds 1 or 4; each of which are about equally protective of the healthy cells against ferroptosis.

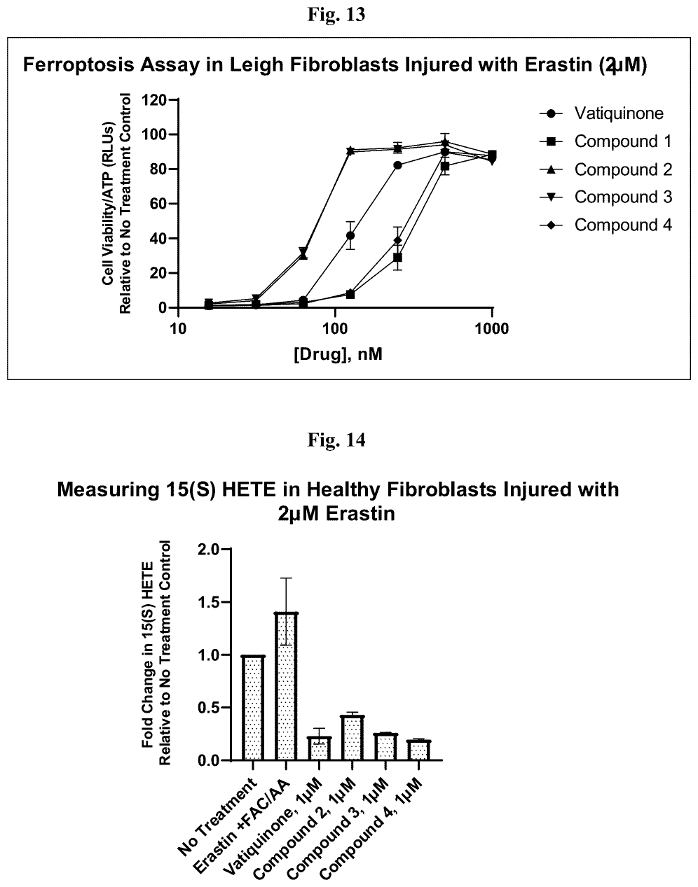

With Reference to FIG. 13, Compounds 1 to 4 and Vatiquinone were examined with respect to whether or not they were protective in fibroblasts from a patient diagnosed with Leigh syndrome. In the assay, Compounds 2 and 3 appear to the superior in their protective effect as compared with Vatiquinone or Compounds 1 or 4. Vatiquinone appears to be superior in its protective effect as compared with Compounds 1 and 4; each of which are about equally protective of the Leigh fibroblasts against ferroptosis.

In summary, each of Compounds 1 to 4 and Vatiquinone appear to exhibit a protective effect with respect to injured cells undergoing ferroptosis without regard to whether the cells are fibroblasts from a healthy individual or those from a patient diagnosed with Leigh Syndrome. In this assay, the protective effect of Compounds 2 and 3 are roughly equivalent and appear to be superior as compared with Vatiquinone or Compounds 1 or 4 in their ability to protect healthy cells or those of a patient diagnosed with Leigh syndrome where the cells are injured with erastin and undergoing ferroptosis.

### F. HETE ELISA/Erastin Assay

In this assay, inhibition of 15-LO by a test article was indirectly measured by quantifying 15(S)-HETE in the supernatant of fibroblasts undergoing ferroptosis. Ferroptosis was induced using erastin at 2 μM concentration. 15(S)-HETE, which is a metabolite of arachidonic acid and generated by activity of 15-LO, was measured using a commercially available ELISA kit. Data were normalized to the “No treatment” group, which represented fibroblasts that were not injured with erastin. As expected, 24-hour incubation with erastin led to increased levels of 15(S)-HETE in the supernatant, as represented by an increase in fold-change compared to the “No treatment” control group.

With Reference to FIG. 14, each of Vatiquinone and Compounds 2 to 4 was tested in fibroblasts from a healthy individual and they appear to exhibit an inhibitory effect on 15-LO when comparing the amount of 15(S)-HETE observed in the absence of a test article (i.e., the bar labeled “Erastin+FAC+AA”). In this assay and under these conditions, it appears that each of Vatiquinone and Compounds 2 to 4 are about equally effective, with Compound 2 perhaps being slightly less effective.

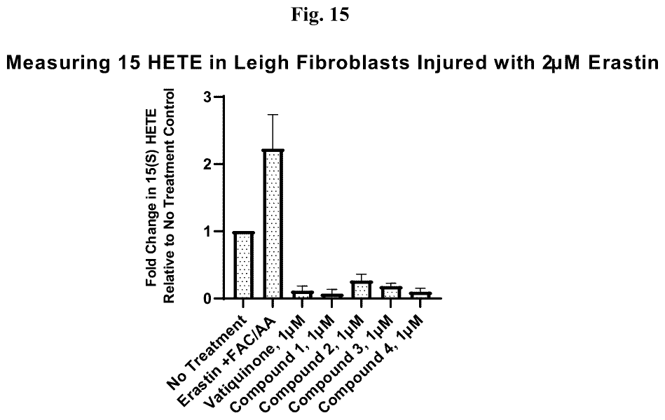

With Reference to FIG. 15, each of Vatiquinone and Compounds 1 to 4 was tested in fibroblasts from a patient diagnosed with Leigh syndrome and in all cases, they appear to exhibit an inhibitory effect on 15-LO when comparing the amount of 15(S)-HETE observed in the absence of a test article (i.e., the bar labeled “Erastin+FAC+AA”). In this assay and under these conditions, it appears that each of Vatiquinone and Compounds 1 to 4 are about equally effective, with Compound 2 perhaps being slightly less effective.

### Example 2: In Vivo Plasma & Tissue Uptake Assays in Mouse and Rat (Compounds 3 & 4)

### I. Assessment of the Pharmacokinetics of Compound 3 and Compound 4 Following Five Daily Subcutaneous Doses to Male C57Bl/6 Mice

### a) Dosing Protocol:

Animals: Male/C57Bl/6 mice/-18-25 grams were received from an approved vendor.
Fasting was not required for this study.
Body weights were recorded prior to dose administration. The volume of each dose delivered (mL/kg) was based on each individual animal's body weight.
This was a multiple dose terminal study dosed once per day over 5 days. Mice were taken down following their 5^{th} and final dose.
Doses were administered in accordance with test facility standard operating procedures. Post-dose flushes were administered as applicable.
All dose syringes were weighed prior to and following dosing to gravimetrically determine the amount of formulation administered.
All animals were observed at dosing and each scheduled collection. Any abnormalities were recorded.
Pre-formulated Compound 3 and Compound 4 were provided by Study sponsor ready to be dosed for each day in accordance with the Study Design set forth in Table 1, below.

TABLE 1
Study Design

| | Number | | Dose | Dose | Dose | | | |
|---|---|---|---|---|---|---|---|---|
| Group | of | Test | Level | Conc. | Volume | Dose | | |
| Number | Animals | Article | (mg/kg) | (mg/mL) | (mL/kg) | Schedule | Vehicle | Route |
| 1 | 12 (3 per | Comp | 20 | 2 | 10 | 5 doses | Kolliphor HS- | SC |
| | timepoint) | 3 | | | | (once per | 15 in PBS, | |
| | | | | | | day) | pH 7.6 | |
| 2 | 12 (3 per | Comp | 60 | 6 | 10 | 5 doses | Kolliphor HS- | SC |
| | timepoint) | 3 | | | | (once per | 15 in PBS, | |
| | | | | | | day) | pH 7.6 | |
| 3 | 12 (3 per | Comp | 60 | 6 | 10 | 5 doses | Kolliphor HS- | SC |
| | timepoint) | 4 | | | | (once per | 15 in PBS, | |
| | | | | | | day) | pH 7.6 | |

SC = subcutaneous

### b) Sample Collection—Post Dosing:

Terminal blood samples were collected via cardiac puncture following inhalation of anesthesia in accordance with test facility standard operating procedures.
Blood samples were collected into tubes with appropriate anticoagulant. The tubes were stored on wet ice until processed to plasma by centrifugation (3500 rpm at 5° C. for 10 minutes) within 20 minutes of collection. Sample aliquots of 100 μL were transferred into individual uniquely labeled matrix tubes and stored at nominal −80° C. until transferred to analytical chemistry for analysis by LC/MS/MS.
Immediately following each terminal blood collection for all groups, animals were sacrificed and perfused with cold PBS. The following tissues were collected: heart and brain in accordance with Table 2.
Plasma and tissue samples were processed for analysis by LC/MS/MS to determine the amounts/concentrations reported in the FIGS. 18-21.

TABLE 2
Sample Collection Design

| | Terminal Blood Collections | Tissue Collections |
|---|---|---|
| Group | Post-Dose (N = 3 per | (n = 3 per dose |
| Number | dose timepoint) | timepoint) |
| 1-3 | 0.5, 2, 8 and 24 hours post | Cold PBS perfusion followed by |
| | 5^{th} and final dose | collection of heart and |
| | | whole brain. |
| | K2 EDTA | N/A |
| Volume/ | Max obtainable (terminal) | N/A |
| Timepoint | | |

### c) Results:

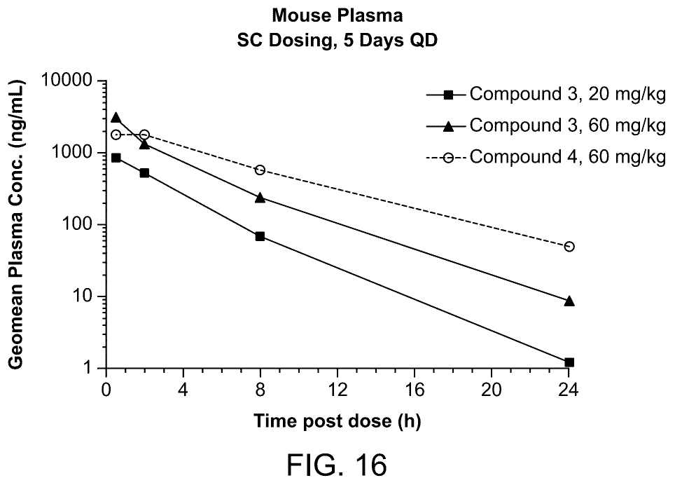

Results are discussed below and presented graphically in FIGS. 16-19. It is also worth noting that administration of Compound 3 and Compound 4 was well tolerated under the concentrations and conditions employed.

### II. An Assessment of the Pharmacokinetics of Compound 3 Following Repeated (5 Days) Subcutaneous Dose Administration to Male Sprague Dawley Rats

### a) Dosing Protocol:

Animals: Rat/Sprague Dawley/Male/Naive 225-250 grams were received from an approved vendor.
Fasting was not required for this study.
Body weights were recorded prior to dose administration. The volume of each dose delivered (mL/kg) was based on each individual animal's body weight.
This was a multiple dose terminal study dosed once per day over 5 days. Rats were taken down following their 5^{th} and final dose.
Doses were administered in accordance with test facility standard operating procedures. Post-dose flushes were administered as applicable.
All dose syringes were weighed prior to and following dosing to gravimetrically determine the amount of formulation administered.
All animals were observed at dosing and each scheduled collection. Any abnormalities were recorded.
Pre-formulated Compounds 3 was provided by Study sponsor ready to be dosed for each day in accordance with the Study Design set forth in Table 3, below.

TABLE 3
Study Design

| | Number | | Dose | Dose | Dose | | | |
|---|---|---|---|---|---|---|---|---|
| Group | of | Test | Level | Conc. | Volume | Dose | | |
| Number | Animals | Article | (mg/kg) | (mg/mL) | (mL/kg) | Schedule | Vehicle | Route |
| 1 | 21 (3 per | Comp | 10 | 2 | 5 | 5 doses | Kolliphor HS- | SC |
| | timepoint) | 3 | | | | (one dose | 15 in PBS, | |
| | | | | | | per day) | pH 7.6 | |

SC = subcutaneous

### b) Sample Collection—Post Dosing:

Terminal blood samples were collected via cardiac puncture following inhalation anesthesia in accordance with test facility standard operating procedures.
Blood samples were collected into tubes with appropriate anticoagulant. Tubes were stored on wet ice until processed to plasma by centrifugation (3500 rpm at 5° C. for 10 minutes) within 20 minutes of collection. Sample aliquots of 100 μL were transferred into individual uniquely labeled matrix tubes and stored at nominal −80° C. until transferred to analytical chemistry for analysis.
Immediately following each terminal blood collection for all groups, animals were sacrificed and perfused with cold PBS. The following tissues were collected: heart and brain, in accordance with Table 4.
Plasma and tissue samples were processed for analysis by LC/MS/MS to determine the amounts/concentrations reported in the Figures.

TABLE 4
Sample Collection Design

| | Terminal Blood Collections | Tissue Collections |
|---|---|---|
| Group | Post-Dose (N = 3 per | (n = 3 per dose |
| Number | dose timepoint) | timepoint) |
| 1 | 0 (24 hours from 4^{th} | Blood, heart and brain |
| | dose), 0.5, 2, 8 and 24 hours | (cortex, striatum, |
| | post 5^{th} and final dose | hippocampus, brain stem). |
| | K2 EDTA | N/A |
| Volume/ | Max obtainable (terminal) | N/A |
| Timepoint | | |

### c) Results:

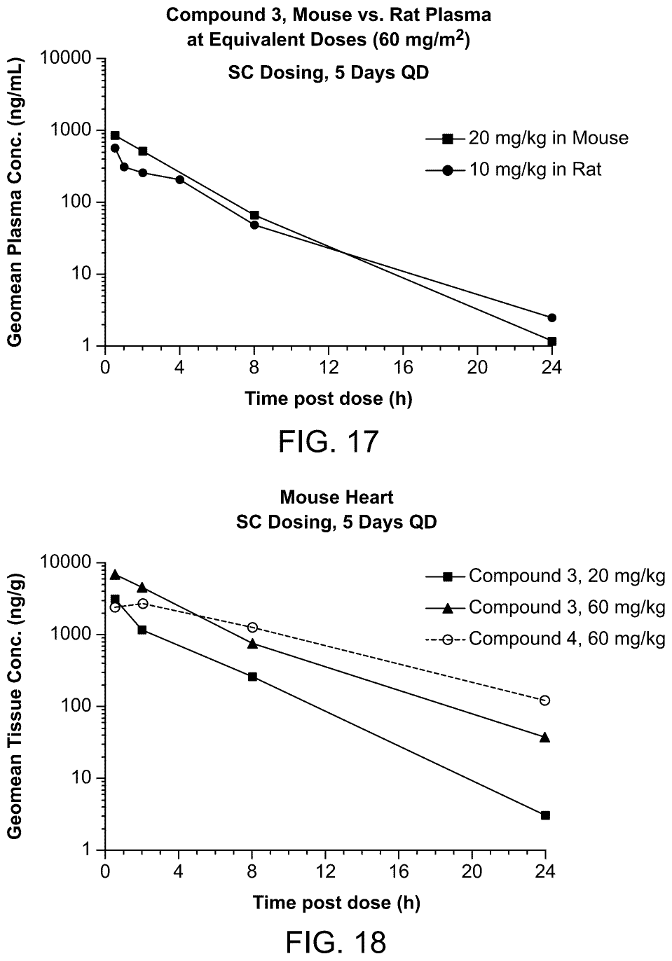

Are discussed below in Section III and presented graphically in FIG. 17. It is also worth noting that administration of Compound 3 was well tolerated under the concentrations and conditions employed.

### III. Relevant Results of the Mouse and Rat PK Experiments

With reference to FIG. 16, the plasma concentrations of Compound 3 (dosed both at 20 mg/kg and 60 mg/kg dosing) and Compound 4 (dosed only 60 mg/kg dosing) are plotted from 30 minutes to 24 hours post-administration. Because the route of administration is subcutaneous injection (SC), the drug (i.e., each of Compounds 3 and 4) needs to migrate to be taken up into the blood stream to provide for potentially efficacious tissue dosing. The data demonstrates that both Compound 3 and Compound 4 migrate into the blood stream of the mouse plasma and that higher dosing elicits a roughly proportional increase in plasma concentrations. Furthermore, the plasma concentrations drop off over time as would be expected as they are cleared from the subject.

In FIG. 17, the data obtained with Compound 3 for plasma concentrations for the 20 mg/kg dose in mouse is graphically compared with the data obtained at 10 mg/kg dosing (also Compound 3) in rats over the period of 30 minutes to 24 hours post administration. Because the comparison is between species (i.e., mouse and rat), these doses are approximately equal if you base them on mg/m² of body area (i.e., in this case 60 mg/m²) of each species. With reference to FIG. 17, the data suggests that uptake of Compound 3 to plasma resulting from SC dosing as well as clearance rate across the two species of mammal is roughly equivalent over the period tested.

Delivery of a drug to the heart can be difficult because the body contains certain barriers (See: Sahoo et al., Targeted Delivery of Therapeutic Agents to the Heart, Nat. Rev Cardiol., (2021) 18(6): 389-399). With reference to FIG. 18, the concentrations of Compound 3 (dosed both at 20 mg/kg and 60 mg/kg dosing) and Compound 4 (dosed only 60 mg/kg dosing) in heart tissue are plotted from 30 minutes to 24 hours post-administration. The data demonstrate that up to microgram quantities of drug per gram of heart tissue were found at up to 2 hours post (after 5 days of) drug administration and that clearance of the drugs from the tissues proceeded over time as expected. These data suggest that efficacious quantities of Compound 3 and Compound 4 can be delivered to heart tissue of mice under these dosing conditions.

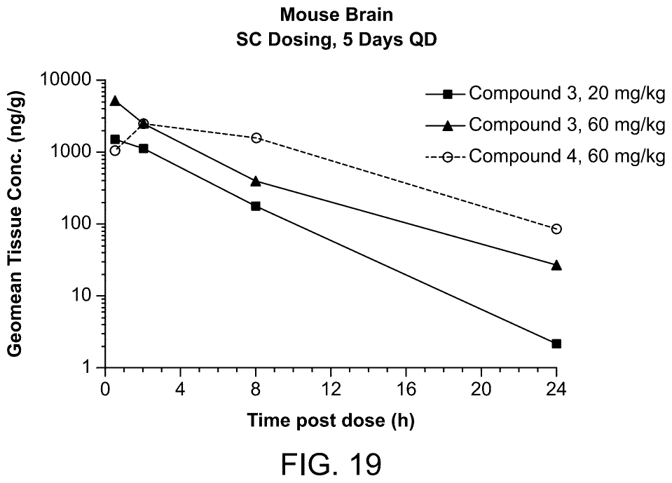

Delivery of a drug to the brain can be difficult because the blood-brain barrier or BBB protects the brain (and other central nervous system (CNS) tissues) against the penetration of certain types of molecules. With reference to FIG. 19, the concentrations of Compound 3 (dosed both at 20 mg/kg and 60 mg/kg dosing) and Compound 4 (dosed only 60 mg/kg dosing) in brain tissue are plotted from 30 minutes to 24 hours post-administration. The data demonstrate that up to microgram quantities of drug per gram of brain tissue were found up to 2 hours post (after 5 days of) drug administration and that clearance of the drugs from the tissues proceeded over time as expected. These data suggest that efficacious quantities of Compound 3 and Compound 4 can be delivered to brain tissue of mice under these dosing conditions.

### d) Summary/Discussion of Results:

Both Compound 3 and Compound 4 were well tolerated at dose levels up to 60 mg/kg in mouse (administered by SC injection).
Compound 3 dose scales well by allometry in rodents (20 mg/kg in mouse ≈10 mg/kg in rat).
Compound 3 plasma exposure is roughly proportional to dose (compare data for 20 mg/kg and 60 mg/kg in mouse).
Tmax was typically later for Compound 4 than Compound 3, and half-life tended to be longer.
Compound 3 exposure (Cmax (maximum observed concentration) and AUC (area under the concentration-time curve)) was higher in heart than in brain; there was no difference in the two tissue exposures for Compound 4.
Based on AUC, heart exposure of Compound 3 was slightly higher that Compound 4; brain exposure of Compound 4 was somewhat higher than Compound 3.
Concentrations achieved in the plasma and heart and brain tissues represent amounts expected to be efficacious for purposes of treatment, prevention, inhibition, amelioration, and/or delay in the onset of Leigh syndrome or the signs or symptoms of Leigh syndrome (e.g., cardiomyopathies (i.e., hypertrophic and dilated cardiomyopathy) and neurodevelopmental and neurodegenerative aspects of the disease (e.g., abnormalities in the brainstem, cerebellum, basal ganglia, oculomotor and cranial nerves and the associated effects resulting in difficulty swallowing, difficulty breathing, difficulty eating, hypotonia, developmental delays, ataxia, dysarthria, dystonia, paralysis and seizures).

### Example 3: In Vivo Assessment of Plasma in Rat of Compound 2 Produced by Various Modes of Administration

### I. Assessment of the Pharmacokinetics of Compound 2 Following a Single Intravenous, Subcutaneous, or Oral Administration to Male Sprague Dawley Rats

### a) Dosing Protocol:

Animals: Rat/Sprague Dawley/Male/Naive 225-250 grams were received from an approved vendor.
Animals were fasted overnight prior to dose administration. Food was returned following the 2 hour sample collection.
Body weights were recorded prior to dose administration. The volume of each dose delivered (ml/kg) was based on each individual animal's body weight.
This was a single dose, serial blood collection study with tissues collected for possible future analysis.
Doses were administered in accordance with test facility standard operating procedures. Post-dose flushes were administered as applicable.
All dose syringes were weighed prior to and following dosing to gravimetrically determine the amount of formulation administered.
All animals were observed at dosing and each scheduled collection. Any abnormalities were recorded.
Pre-formulated Compound 2 and Vatiquinone were provided by Study sponsor ready to be dosed in accordance with the Study Design set forth in Table 5, below.

TABLE 5
Study Design

| | Number | | Dose | Dose | Dose | | | | Post |
|---|---|---|---|---|---|---|---|---|---|
| Group | of | Test | Level | Conc. | Volume | Dose | | | Dose |
| Number | Animals | Article | (mg/kg) | (mg/mL) | (mL/kg) | Schedule | Vehicle | Route | Flush |
| 1 | 3 | Compound | 1 | 0.2 | 5 | Single | Kolliphor | IV | 0.5 |
| | | 2 | | | | Dose | HS-15 | | mL |
| | | | | | | | in PBS, | | |
| | | | | | | | pH 7.6 | | |
| 2 | 3 | Compound | 10 | 2 | 10 | Single | Kolliphor | SC | N/A |
| | | 2 | | | | Dose | HS-15 | | |
| | | | | | | | in PBS, | | |
| | | | | | | | pH 7.6 | | |
| 3 | 3 | Compound | 10 | 2 | 10 | Single | Kolliphor | PO | N/A |
| | | 2 | | | | Dose | HS-15 | | |
| | | | | | | | in PBS, | | |
| | | | | | | | pH 7.6 | | |
| 4 | 3 | Vatiquinone | 10 | 2 | 10 | Single | 90% corn | PO | N/A |
| | | | | | | Dose | oil + 1-% | | |
| | | | | | | | DMSO | | |

IV = Intravenous,
SC = subcutaneous,
PO = oral,
N/A = Not Applicable

### b) Sample Collection—Post Dosing:

Blood samples were collected via jugular vein catheter (JVC) and transferred to tubes with appropriate anticoagulant. The tubes were stored on wet ice until processed to plasma by centrifugation (3500 rpm at 5° C. for 10 minutes) within 20 minutes of collection. Sample aliquots of 100 μL were transferred into individual uniquely labeled matrix tubes containing 10 μL of 5% (v/v) formic acid aqueous solution and stored at nominal −80° C. until analyzed by LC/MS/MS.
Immediately following final serial blood collection (24 hours, JVC), animals were sacrificed and perfused with cold PBS. The following tissues were collected: heart and brain (stored for possible future analysis), in accordance with Table 6.
Plasma and tissue samples were processed for analysis by LC/MS/MS to determine the amounts/concentrations reported in the Figures.

TABLE 6
Sample Collection Design

| | Terminal Blood Collections | Tissue Collections |
|---|---|---|
| Group | Post-Dose (N = 3 per | (n = 3 per dose |
| Number | dose timepoint) | timepoint) |
| 1-4 | 0.5, 2, 8 and 24 hours post | Whole brain and heart (stored |
| | dose | for future analysis). |
| | K2 EDTA | N/A |
| Volume/ | About 250 μL | N/A |
| Timepoint | | |

### c) Results:

Results are preliminary and under continued review. However, the following observations were made with respect to the results of this experiment using Compound 2.
Compound 2 was effectively administered (i.e., the data suggests that an efficacious dose can be delivered to the subject) without regard to the mode of administration (e.g., intravenous, subcutaneous or oral administration).
Regardless of the mode of administration, Compound 2 was well tolerated at the dosing levels examined in this study.

### Example 4: In Vitro Comparison of Leigh Fibroblasts Treated with Compound 3 when Treated with RSL3 to Induce Glutathione Stress

### Methods:

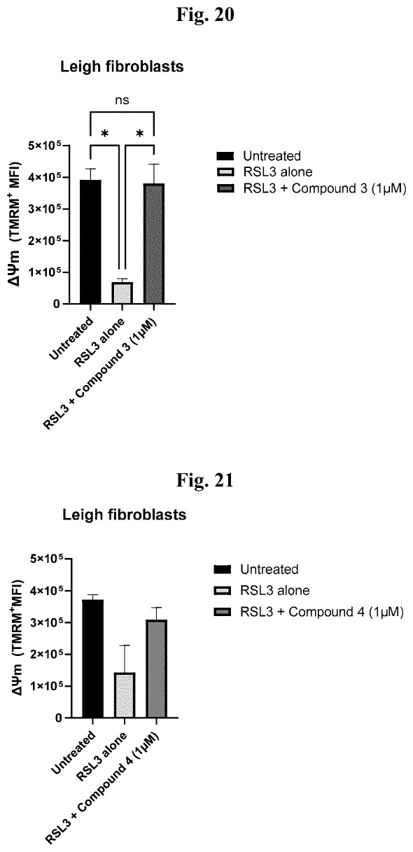

Leigh fibroblasts (GM03672) were seeded in a 24-well, tissue culture-treated plate in complete MEM (MEM+15% FBS+1% penicillin/streptomycin) at a density of 1×10⁵ cells per 500 μL and incubated overnight at 37° C. at 5% CO₂. Cells were treated for 4 hours at 37° C. with either 1 μM RSL3, 1 μM RSL3 with 1 μM Compound 3 or media for untreated cells. Following the incubation, supernatant was removed and cells were treated with 0.25% trypsin for 5 min at 37° C. Trypsin was neutralized with an equal part MEM. Cells were dislodged with gentle pipetting and pelleted at 300×g for 5 minutes. Pellets were resuspended in 500 μL Fluobrite DMEM and cells were transferred to a 96-well, v-bottom plate. Cells were stained with 20 nM MitoTracker Green and 100 nM tetramethylrhodamine methyl ester (TMRM) Samples were incubated for 25 minutes at 37° C. Each well was mixed immediately prior to analysis to resuspend settled cells. Fluorescence intensity was measured on the BD Accuri C6 Plus Flow Cytometer. A minimum of 10,000 events per sample was collected. Data analysis was performed using FloJo vs. 10. Gating was performed by establishing an initial forward scatter vs. side scatter gate. MitoTracker Green positive, TMRM positive cells were identified through subsequent gating. Median fluorescence intensity (MFI) of TMRM fluorescence was determined. Using GraphPad Prism 10, statistical significance between groups was determined by One-way ANOVA. A p-value of less than 0.05 was considered significant. Results are presented graphically in FIG. 20.

### Results:

With reference to FIG. 20, the results for untreated Leigh fibroblasts and those treated with both RSL3 and Compound 3 (at 1 μM concentration) are comparable (i.e., essentially the same). However, those Leigh fibroblasts treated with RSL3, alone, show a significant reduction in mitochondrial membrane potential. The effect of RSL3 treatment (when treated alone) on the Leigh fibroblasts is consistent with the induction of ferroptosis or glutathione stress in the cell line, a well-known effect of RSL3 treatment. However, the result that occurs when the Leigh fibroblasts are treated with both RSL3 and Compound 3 suggest that Compound 3 (at 1 μM concentration) is protective of the Leigh fibroblasts from the loss of mitochondrial membrane potential associated with glutathione stress or ferroptosis otherwise induced by RSL3.

With reference to FIG. 21, the results for untreated Leigh fibroblasts and those treated with both RSL3 and Compound 4 (at 1 μM concentration) are comparable (i.e., essentially the same). However, Leigh fibroblasts treated with RSL3, alone, show a significant reduction in mitochondrial membrane potential. The effect of RSL3 treatment (when treated alone) on the Leigh fibroblasts is consistent with the induction of ferroptosis or glutathione stress in the cell line, a well-known effect of RSL3 treatment. However, the result that occurs when the Leigh fibroblasts are treated with both RSL3 and Compound 4 suggest that Compound 4 (at 1 μM concentration) is protective of the Leigh fibroblasts from the loss of mitochondrial membrane potential associated with glutathione stress or ferroptosis otherwise induced by RSL3.

### Example 5: Compound 4 Pharmacokinetics and Tissue Exposure Following Five Daily Subcutaneous Doses to Male C57BL/6 Mice

This example demonstrates the pharmacokinetics and dose proportionality of exposure in plasma and tissues (heart and brain) in mice dosed with 60 mg/kg Compound 4 or 180 mg/kg Compound 4, and provides a comparison of 60 mg/kg Compound 4 using 15% Kolliphor ELP to 60 mg/kg Compound 4 using 5% Kolliphor ELP.

### Study Design Details:

Animals were allowed to acclimate to the test facility for at least two days prior to the beginning of the study.
Study animals were male C57BL/6 mice weighing approximately 18-25 grams each.
Fasting was not required for the study.
Body weights were recorded prior to dose administration. The volume of each dose delivered (mL/kg) was based on each individual animal's body weight.
This was a multiple dose serial PK dosed subcutaneously once daily over 5 days. Compound 4 was administered to two groups: Group 1 at 60 mg/kg and Group 2 at 180 mg/kg (6 and 18 mg/mL, 15% Kolliphor ELP in PBS, 10 mL/kg; N=3/group). Serial PK timepoints were collected following the 5^{th} and final dose. Mice were taken down following their 5^{th} and final dose for blood and tissue collection.
All dose syringes were weighed prior to and following dosing to gravimetrically determine the amount of formulation administered.
All animals were observed at dosing and each scheduled collection.
The same design was used for Group 3 mice administered 60 mg/kg using 5% Kolliphor ELP SC once daily over 5 days, except that 3 mice were taken down at each of the four PK timepoints (n=12).

TABLE 7
Study Design

| | Number | | Dose | Dose | Dose | | | |
|---|---|---|---|---|---|---|---|---|
| Group | of | Test | Level | Conc. | Volume | Dose | | |
| Number | Animals | Article | (mg/kg) | (mg/mL) | (mL/kg) | Schedule | Vehicle | Route |
| 1 | 3M | Compound | 60 | 6 | 10 | 5 doses | 15% | SC |
| | | 4 | | | | (one dose | Kolliphor | |
| | | | | | | per day) | ELP in PBS, | |
| | | | | | | | pH 7.6 | |
| 2 | 3M | Compound | 180 | 18 | 10 | 5 doses | 15% | SC |
| | | 4 | | | | (one dose | Kolliphor | |
| | | | | | | per day) | ELP in PBS, | |
| | | | | | | | pH 7.6 | |

TABLE 8
Formulation Design

| | | | | Residual |
|---|---|---|---|---|
| Test | Compound | Test Article | Formulation | Formulation |
| Article | information | Storage | Storage | Storage |
| Compound | MW = 402.50 | −20° C. | 20-25° C. | 20-25° C. |
| 4 | g/mol; | | | |

### Sample Collection Details:

Serial blood samples were collected via tail vein snip.
Blood samples were collected into tubes with anticoagulant. Tubes were stored on wet ice until processed to plasma by centrifugation (3500 rpm at 5° C. for 10 minutes) within 20 minutes of collection. Sample aliquots of 20 μL were transferred into individual uniquely labeled matrix tubes and stored at nominal −80° C. until transferred to analytical chemistry for analysis.
Immediately following each terminal blood collection for all groups, animals were sacrificed and perfused with cold PBS. Heart and brain tissues were collected.
   Perfusion methodology: The chest was opened lengthwise using scissors to expose the thoracic organs. A scalpel was used to cut a small hole into the left ventricle just large enough to insert a cannula tip. The cannula was attached to an appropriately sized tubing and syringe prefilled with cold PBS and primed to remove any air. The tip of the cannula was inserted into the left ventricle and directed into the ascending aorta. Hemostats were used to help keep the cannula in place during perfusion. A second hole was made in the right atrium to allow for the escape of fluid during the return circulation. The syringe attached to the cannula was manually pushed or placed into a pump programmed to infuse at 10 mL/min. Animals were perfused for a minimum of 3 minutes or more if needed (until the exiting fluid was clear).

TABLE 9
Sample Collection Design

| | | Tissue Collections |
|---|---|---|
| Group | | (24 hours post 5^{th} |
| Number | Blood Collections | and final dose) |
| 1 | 2 and 24 hours post 5^{th} | Cold PBS perfusion |
| | and final dose | followed by collection |
| 2 | Pre 5^{th} dose, 2, 8, and | of heart and whole |
| | 24 hours post 5^{th} and | brain |
| | final dose | |
| Anticoagulant | K2 EDTA | NA |
| Volume/ | 60 μL (*terminal 24 hr post 5^{th} | NA |
| Timepoint | dose sample is maximum draw) | |
| | Terminal Blood Collections | Tissue Collections |
| Group | Post-Dose (n = 3 per | (n = 3 per |
| Number | timepoint) | timepoint) |
| 3 | 0.5, 2, 8 and 24 hours post | Cold PBS perfusion |
| | 5^{th} and final dose | followed by collection |
| | | of heart and whole brain |
| Anticoagulant | K2 EDTA | NA |
| Volume/ | Max obtainable (terminal) | NA |
| Timepoint | | |

### Analyses:

### Groups 1 and 2

N=3/group;
Collections relative to the final dose:
   Plasma: 60 mg/kg at 2 and 24 hr; 180 mg/kg at predose, 2, 8, and 24 hr
   Heart and Brain: 24 hr (terminal, PBS-perfused);
RGA 2 bioanalysis.

### Group 3

N=3/timepoint;
Collections relative to the final dose:
   Plasma: 0.5, 2, 8, and 24 hr (terminal)
   Heart and Brain: 0.5, 2, 8, and 24 hr (terminal, PBS-perfused)
RGA 2 bioanalysis.

TABLE 10
Sample Analyses
Sample Processing

| | |
|---|---|
| Extraction Volume (μL): | 10 |
| Extraction Method: | Protein Precipitation |

Sample Analysis
LC Conditions:

| Column Id. & | Waters Acquity; HSS T3; 1.8 μm; | Time | % | Flow |
|---|---|---|---|---|
| Dimensions: | 50 × 2.1 mm | (sec) | MPB | (mL/min) |
| Temperature (° C.) | 45 | 0.00 | 65 | 0.800 |
| Mobile Phase A | 10 mM AmmAcet in 95:5:0.1 | 0.20 | 65 | 0.800 |
| | H2O:ACN:FA | | | |
| Mobile Phase B | 10 mM AmmAcet in 50:50:0.1 | 1.58 | 100 | 0.800 |
| | ACN:MeOH:FA | | | |
| Needle Rinse 1 | 50:25:25 IPA:Acetone:MeOH | 1.60 | 100 | 0.800 |
| Needle Rinse 2 | 90:10:0.1 H2O:MeOH:FA | 2.00 | 100 | 0.800 |
| | | 2.02 | 65 | 0.800 |
| | | 2.50 | 65 | 0.800 |

MS Conditions:

| | |
|---|---|
| MS/MS: | API-6500+ |
| Ionization Method: | Electrospray |
| Positive/Negative Ion: | Positive |
| Resolution: | Unit/Unit |
| Source Temperature (° C.): | 550 |
| Transitions (m/z): | Compound ID: Compound 4 402.100/163.100 Da |
| | Int Std ID: Glyburide 492.200/367.200 Da |

Data Analysis

| | |
|---|---|
| Acceptance Criteria | ±20%, ±25% at LLOQ |
| Regression Type | Linear (1/(x * x)) |
| Accepted Curve Range | 0.500-250 ng/mL |
| Carryover | Not Observed |

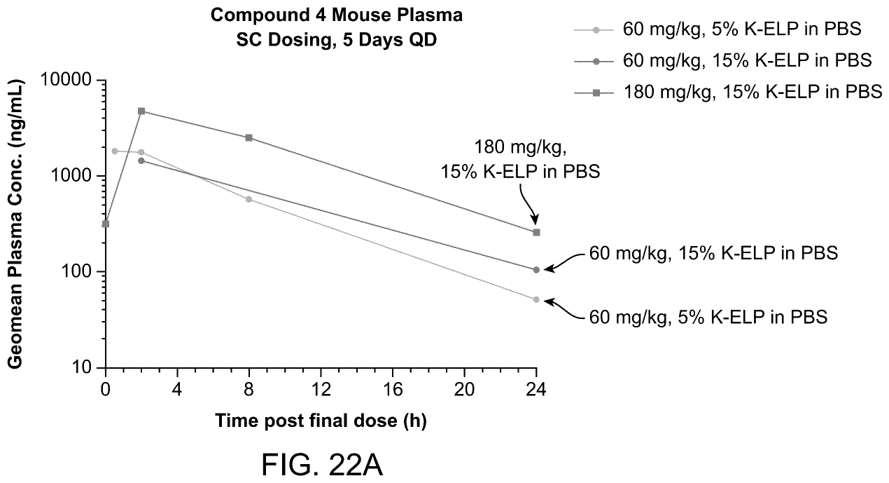

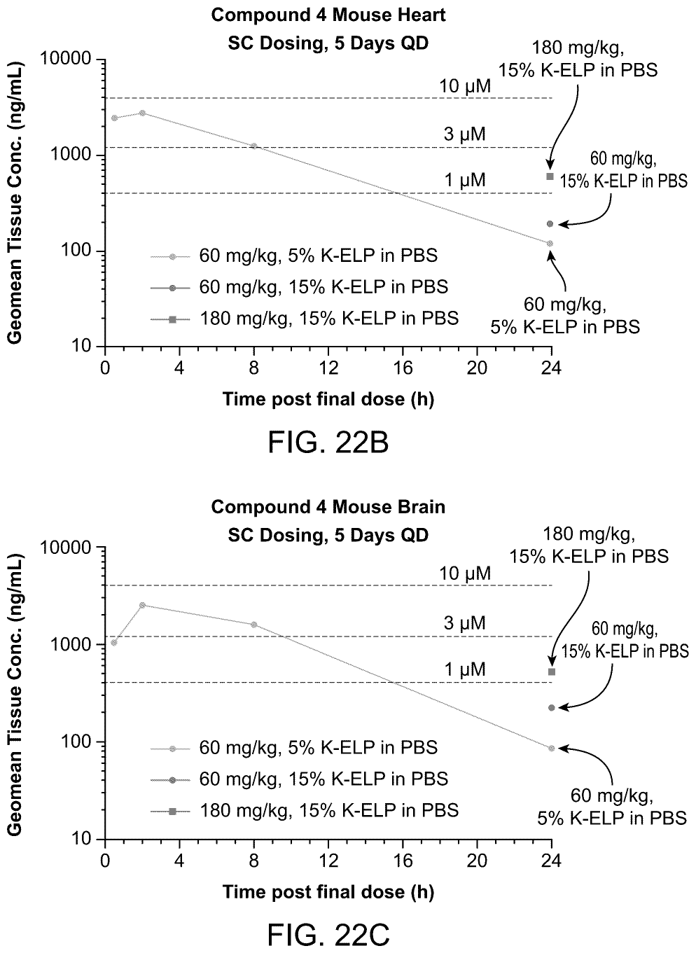

Results—Mouse Plasma PK Profile after 5 Days (FIG. 22A; Tables 11-13):
60 and 180 mg/kg SC was well tolerated over 5 days (10 mL/kg QD, 15% Kolliphor ELP in PBS);
5% and 15% Kolliphor ELP produced similar Compound 4 plasma concentrations at 60 mg/kg;
Tmax ˜0.5-2 hr;
Steady-state achieved within 5 days;
Compound 4 plasma exposure was approximately proportional to dose.

TABLE 11
Compound 4 Mouse Plasma Concentrations (Groups 1 and 2)

| Compound 4 Concentration in C57BL/6 Mouse Plasma | | | | | | | | | |
|---|---|---|---|---|---|---|---|---|---|
| | | | Calculated | | | | | | |
| | | | Concentration | | | | | | |
| | | | (ng/mL) | | | | | | |
| | | Timepoint | Animal ID | | | | | | |
| Group | Dose | (Hours) | 1 | 2 | 3 | Mean | SD | % CV | Geomean |
| 1 | 60 mg/kg; SC | 2 | 1140 | 1610 | 1570 | 1440 | 261 | 18.1% | 1423 |
| | | 24 | 105 | 93.7 | 118 | 106 | 12.2 | 11.5% | 105 |
| | | | 4 | 5 | 6 | Mean | SD | % CV | Geomean |
| 2 | 180 mg/kg; SC | Predose | 367 | 372 | 226 | 322 | 82.9 | 25.8% | 314 |
| | | 2 | 3800 | 5550 | 5050 | 4800 | 901 | 18.8% | 4740 |
| | | 8 | 2320 | 2690 | 2480 | 2497 | 186 | 7.4% | 2492 |
| | | 24 | 345 | 235 | 218 | 266 | 68.9 | 25.9% | 260 |

TABLE 12
Compound 4 Mouse Plasma Concentrations (Group 3)
Concentration in C57BL/6 Mouse Plasma

| | | Time- | | Calculated | |
|---|---|---|---|---|---|
| | | point | Animal | Concentration | |
| Group | Dose | (Hr) | ID | (ng/mL) | Geomean |
| 3 | 60 mg/kg, SC | 0.5 | 25 | 1580 | |
| | | | 26 | 2240 | 1786 |
| | | | 27 | 1610 | |
| | | 2 | 28 | 1900 | |
| | | | 29 | 2130 | 1778 |
| | | | 30 | 1390 | |
| | | 8 | 31 | 702 | |
| | | | 32 | 524 | 578 |
| | | | 33 | 526 | |
| | | 24 | 34 | 56.9 | |
| | | | 35 | 35.9 | 50.7 |
| | | | 36 | 63.9 | |

TABLE 13
Plasma PK

| | Dose | Tmax | Cmax | Cmax/Dose | AUClast | AUClast/Dose | C24 | C24/Dose |
|---|---|---|---|---|---|---|---|---|
| Formulation | (mg/kg) | (h) | (ng/mL) | (kg*ng/mL/mg) | (h*ng/mL) | (h*kg*ng/ml/mg) | (ng/ml) | (kg*ng/ml/mg) |
| 5% Kolliphor | | | | | | | | |
| ELP in PBS | 60 | 0.5 | 1810 | 30.2 | 15400 | 257 | 52.2 | 0.871 |
| 15% Kolliphor | | | | | | | | |
| ELP in PBS | 60 | 2.0 | 1440 | 24.0 | NC | NC | 106 | 1.76 |
| | 180 | 2.0 | 4800 | 26.7 | 49100 | 273 | 266 | 1.48 |

NC = Not calculated

Results—Mouse Tissue Exposure Profile after 5 Days (FIGS. 22B-22C; Tables 14-16):
Similar Compound 4 concentrations found in heart and brain;
C24 (trough) exposure was approximately proportional to dose;
180 mg/kg QD maintained tissue concentrations >1 μM.

TABLE 14
Compound 4 Concentration in Mouse Tissue (Groups 1 and 2)
Compound 4 Concentration in C57BL/6 Mouse Tissue

| | | | Calculated | | | | | | |
|---|---|---|---|---|---|---|---|---|---|
| | | | Concentration (ng/g) | | | | | | |
| | | | Animal ID | | | | | | |
| Group | Dose | Tissue | 1 | 2 | 3 | Mean | SD | % CV | Geomean |
| 1 | 60 mg/kg; SC | Brain | 196 | 218 | 265 | 226 | 35.2 | 15.6% | 225 |
| | | Heart | 200 | 109 | 326 | 212 | 109 | 51.5% | 192 |
| | | | 7 | 8 | 9 | Mean | SD | % CV | Geomean |
| 2 | 180 mg/kg; SC | Brain | 697 | 504 | 387 | 529 | 157 | 29.6% | 514 |
| | | Heart | 922 | 565 | 419 | 635 | 259 | 40.7% | 602 |

TABLE 15
Compound 4 Concentration in Mouse Tissue (Group 3)
Compound 4 Concentration in C57BL/6 Mouse Tissue (ng/g)

| | | | | Calculated | | | | | |
|---|---|---|---|---|---|---|---|---|---|
| | | | | Concentration | | | | | |
| | | Timepoint | Animal | (ng/g) | | Geomean | | | |
| Group | Dose | (Hr) | ID | Heart | Brain | Heart | μM | Brain | μM |
| 3 | 60 mg/kg | 0.5 | 25 | 1680 | 932 | | | | |
| | | | 26 | 3130 | 1340 | 2457 | 6.10 | 1043 | 2.59 |
| | | | 27 | 2820 | 909 | | | | |
| | | 2 | 28 | 2480 | 2960 | | | | |
| | | | 29 | 3810 | 2460 | 2762 | 6.86 | 2513 | 6.24 |
| | | | 30 | 2230 | 2180 | | | | |
| | | 8 | 31 | 745 | 1860 | | | | |
| | | | 32 | 1650 | 1330 | 1261 | 3.13 | 1592 | 3.95 |
| | | | 33 | 1630 | 1630 | | | | |
| | | 24 | 34 | 133 | 95.3 | | | | |
| | | | 35 | 99.7 | 61.8 | 120.5 | 0.30 | 85.5 | 0.21 |
| | | | 36 | 132 | 106 | | | | |

TABLE 16
Tissue Exposure

| | | | Geomean | |
|---|---|---|---|---|
| Tis- | | Dose | C24 | C24/Dose |
| sue | Formulation | (mg/kg) | (ng/g) | (kg*ng/g/mg) |
| Heart | 5% Kolliphor ELP in PBS | 60 | 121 | 2.01 |
| | 15% Kolliphor ELP in PBS | 60 | 192 | 3.20 |
| | | 180 | 602 | 3.34 |
| Brain | 5% Kolliphor ELP in PBS | 60 | 85.5 | 1.42 |
| | 15% Kolliphor ELP in PBS | 60 | 225 | 3.74 |
| | | 180 | 514 | 2.86 |

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a nonlimiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

1. A method for treating, preventing, ameliorating, inhibiting, or delaying the onset of Leigh syndrome, or its associated signs and/or symptoms, in a subject diagnosed with Leigh syndrome in need thereof, comprising administering to said subject a therapeutically effective amount of Compound 1, Compound 2, Compound 3, Compound 4, or a mixture of any two or more of Compounds 1 to 4, or pharmaceutically acceptable salts, stereoisomers, mixtures of stereoisomers, tautomers, hydrates, and/or solvates thereof, wherein Compounds 1 to 4 have the following structures:

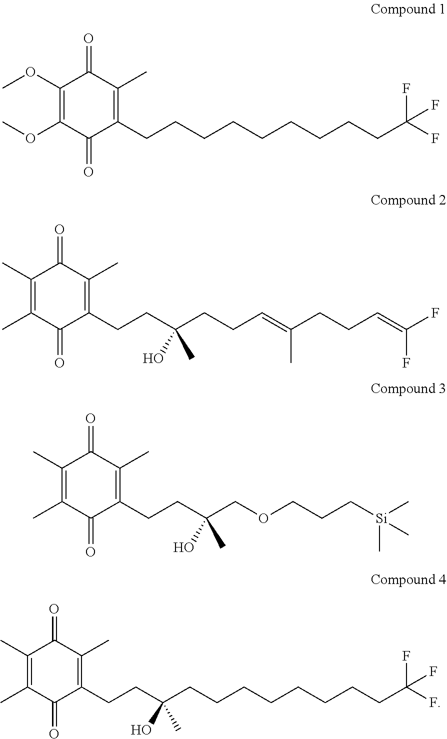

2. The method of claim 1, wherein the signs or symptoms of Leigh syndrome include difficulty swallowing, difficulty breathing, difficulty eating, hypotonia, lactic acidosis, developmental delays, cardiomyopathy, seizures, ataxia, dysarthria, dystonia, paralysis, impairment of respiratory and kidney function, color blindness and/or vision loss.

3. (canceled)

4. The method of claim 1, wherein the Compound(s) is/are administered daily for 6 weeks or more.

5. The method of claim 1, wherein the Compound(s) is/are administered orally, topically, intranasally, systemically, intravenously, subcutaneously, intraperitoneally, intradermally, intraocularly, ophthalmically, intrathecally, intracerebroventricularly, iontophoretically, transmucosally, intravitreally, or intramuscularly.

6. The method of claim 1, wherein administration of the Compound(s), to the subject treats, prevents, ameliorates, inhibits, or delays the onset of a cardiomyopathy in the subject.

7. The method of claim 6, wherein the cardiomyopathy is hypertrophic cardiomyopathy, left ventricular hypertrophy, dilated cardiomyopathy, pericardial effusions and/or an arrhythmia/conduction abnormality.

8. The method of claim 6, wherein the cardiomyopathy is hypertrophic cardiomyopathy.

9. The method of claim 1, wherein administration of the Compound(s) to the subject lowers lactic acid levels in the subject's blood, urine, or cerebrospinal fluid (CSF).

10. The method of claim 1, wherein administration of the Compound(s), to the subject treats, prevents, ameliorates, inhibits, or delays the onset of multi-organ and/or central nervous system (CNS) damage, including lesions in the brainstem, basal ganglia, and spinal cord of the subject.

11. The method of claim 1, wherein administration of the Compound(s) to the subject treats, prevents, ameliorates, inhibits, or delays the onset of color blindness and/or vision loss of the subject.

12. The method of claim 1, wherein administration of the Compound(s), to the subject, increases the lifespan of the subject.

13. The method of claim 1, wherein administration of the Compound(s) to the subject treats, prevents, ameliorates, inhibits, or delays the onset of seizures, ataxia, dysarthria, dystonia and/or paralysis of the subject.

14. The method of claim 1, wherein the subject is human.

15.-42. (canceled)
